(19) United States
(12) Patent Application Publication
(10) Pub. No.: US 2015/0307949 A1
(43) Pub. Date: Oct. 29, 2015

(54) IDENTIFICATION AND ERADICATION OF INSECT PESTS

(71) Applicants: David Bailey, Greenwich (GB); Susan Seal, Greenwich (GB); Mitul Patel, Greenwich (GB); Carl Collins, Greenwich (GB); John Colvin, Greenwich (GB)

(72) Inventors: David Bailey, Greenwich (GB); Susan Seal, Greenwich (GB); Mitul Patel, Greenwich (GB); Carl Collins, Greenwich (GB); John Colvin, Greenwich (GB)

(73) Assignee: University of Greenwich, Greenwich (GB)

(21) Appl. No.: 13/703,476

(22) PCT Filed: Jun. 13, 2011
(86) PCT No.: PCT/GB2011/051097

§ 371 Date: May 3, 2013

(30) Foreign Application Priority Data
Jun. 11, 2010 (GB) 1009816.8

(51) Int. Cl. C12Q 1/68; G01N 33/68

(52) U.S. Cl. CPC C12Q 1/6888; G01N 33/68; G01N 2333/43569; G01N 2333/988; G01N 2333/99; G01N 2405/00; C12Q 2600/124; C12Q 2600/156

(57) ABSTRACT

A method of identifying the biotype (or clade) of a whitefly comprising using at least one of: (a) an hsp90 gene, polypeptide or protein, (b) an enolase gene, polypeptide or protein, (c) a fructose bisphosphate aldolase gene, polypeptide or protein, (d) a triose phosphate isomerase gene, polypeptide or protein, or (e) a vitellogenin gene, polypeptide or protein, of the whitefly. A kit for identifying B. tabaci biotype Asia is also provided as well as isolated polynucleotides and polypeptides having SEQ ID 10 NOs: 1-9, and polynucleotides and antibodies that bind SEQ ID NOs: 1-9.

The present invention relates to improvements in or relating to identification and eradication of insect pests such as those which spread disease, or reduce crop yield. It especially relates to whitefly and their biotypes (or clades), and particularly to genetic sequences, polypeptides, proteins and polymorphisms associated with individual whitefly biotypes which can be used for biotype identification or as targets for agrochemical discovery. More particularly, the present invention relates to sequences, polypeptides, proteins and polymorphisms associated with Bemisia tabaci biotype (sequence Glade) Asia 1 whitefly and its component proteins.

Whitefly is an economically-significant agricultural pest that causes damage to crops by feeding on phloem sap (reducing crop yield) and by acting as a vector for plant viruses. Whitefly infestations are generally controlled by widespread application of chemical insecticide. This control method, however, has led to growing insecticide resistance within whitefly populations, necessitating the development of new insecticides acting through novel biochemical mechanisms. The use of ineffective insecticides in areas where pests are already showing resistance is potentially damaging since it wastes insecticide, causes delay in treating the infestation and can accelerate the spread of super-resistance to insecticides. Moreover, the development of such super-pests is a major threat to food security.

One way to address the problem of insecticide resistance is to use insecticides that are selective for particular species or populations. Historically, when identifying insect taxa, morphology has been used to separate species. However, harmful members of an insect species, for example those which are resistant to agrochemicals and therefore pose a threat to food security, are morphologically indistinguishable from less harmful members of the species, for example those that are insecticide susceptible.

One way of characterising harmful insects, including harmful whitefly, is by using biochemical and molecular polymorphisms associated with harmful phenotypes. Over twenty-four whitefly biotypes can be characterised by genetic variations, using the mitochondrial gene cytochrome c oxidase I (mtCOI). Individual whitefly biotypes are also known to differ in characteristics such as their ability to transmit viral diseases.

The present inventors have characterised the coding sequences of a number of whitefly proteins using the B. tabaci Asia 1 biotype whitefly. These proteins include: the heat shock protein 90 (hsp90), the protein vitellogenin, and several metabolic enzymes, including enolase, triose phosphate isomerase, and fructose bisphosphate aldolase. Hsp90 is a molecular chaperone that is upregulated in response to stress. Vitellogenin is a glycolipoprotein involved in egg formation in nearly all oviparous species. It is also associated with food storage reservoirs produced by honey bees. Metabolic enzymes of the type characterised in the present invention catalyze various stages in cellular energy production. Some of the proteins that have been characterised, such as hsp90, are found in all eukaryotic cells. Proteins such as vitellogenin, on the other hand, are found only in specific eukaryotic cells.

As part of the present invention, genes corresponding to the characterised proteins were compared to genes found in other insect species and in other whitefly biotypes. In particular, Apis mellifera, Aedes aegypti, Acyrthosiphon pisum, Drosophila melanogaster, Anopheles gambiae, Tribolium castaneum, Bemisia tabaci biotype ZHJ1, Bemisia tabaci biotype B and Bemisia tabaci biotype Q were used in comparative studies. Relevant coding sequences are available as Genbank Accession Numbers:

### Hsp90

HM013713 (Bemisia tabaci biotype ZHJ1)
HM013710 (Bemisia tabaci biotype Q)
DQ093381.1; DQ093380.1; EU934241 (Bemisia tabaci biotype B)
NM_{—}001160064.1 (Apis mellifera)
XM_{—}001943137.1 (Acyrthosiphon pisum)
NM_{—}079175.2 (Drosophila melanogaster)
XM_{—}308800.4 (Anopheles gambiae)
NM_{—}001100597.1 (Tribolium castaneum)

### Enolase

XM_{—}001653700.1 (Aedes aegypti)
XM_{—}317672.4 (Anopheles gambiae)
DQ864194.1 (Drosophila melanogaster)
XM_{—}962466.2 (Tribolium castaneum)
XM_{—}001948126.1 (Acyrthosiphon pisum)

### Fructose Bisphosphate Aldolase

XM_{—}312372.4 (Anopheles gambiae)
XM_{—}001651373.1 (Aedes aegypti)
DQ864133.1 (Drosophila melanogaster)
X60064.1 (Drosophila melanogaster)
XM_{—}001951482.1 (Acyrthosiphon pisum)
XM_{—}623339.2 (Apis mellifera)

### Triose Phosphate Isomerase

XM_{—}001655536.1 (Aedes aegypti)
X57576.1 (Drosophila melanogaster)
XM_{—}970400.2 (Tribolium castaneum)
NM_{—}001097154.1 (Apis mellifera)
NM_{—}001162204.1 (Acyrthosiphon pisum)

The present invention seeks to provide methods and means for identifying specific whitefly biotypes—so that infestations can be monitored and so that selective application of insecticides can be used to control infestation. The present invention further seeks to provide protein targets for insecticide discovery.

In its broadest aspect, the present invention provides genetic sequences of B. tabaci biotype (clade) Asia 1 proteins. Polynucleotide sequences, polypeptide sequences, proteins and polymorphisms associated with individual whitefly biotypes are provided.

In one aspect, methods are provided for using whitefly genetic information or genetic variation to identify B. tabaci biotype Asia 1 whitefly.

In another aspect, whitefly proteins for use in developing agents, such as agrochemicals that target specific whitefly proteins, are provided.

In a yet further aspect, variations in gene and protein sequences are provided for use in discovery of insect-, whitefly- or biotype-specific insecticides.

Genetic sequences, polypeptides, proteins and polymorphisms associated with whitefly biotypes provided by the present invention are used as targets for agrochemical discovery. The present invention also provides chemical agents that recognise whitefly polypeptides and methods of screening for such agents. The chemical agents preferably have insecticide activity.

According to the present invention, there is provided a method of identifying the biotype of a whitefly, the method comprising using one or a combination of:
(a) an hsp90 gene, polypeptide or protein,
(b) an enolase gene, polypeptide or protein,
(c) a fructose bisphosphate aldolase gene, polypeptide or protein,
(d) a triose phosphate isomerase gene, polypeptide or protein, or
(e) a vitellogenin gene, polypeptide or protein, of the whitefly.

In one embodiment, the method comprises reacting at least one of:
(a) the hsp90 protein with an agent that recognises the polypeptide of SEQ ID NO: 2,
(b) the enolase protein with an agent that recognises the polypeptide of SEQ ID NO: 4,
(c) the fructose bisphosphate aldolase protein with an agent that recognises the polypeptide of SEQ ID NO: 6, or
(d) the triose phosphate isomerase protein with an agent that recognises the polypeptide of SEQ ID NO: 8,

Preferably, the agent is an antibody.

In one embodiment, the method comprises at least one of:
(a) determining the amino acid sequence of the hsp90 protein of the whitefly and comparing the amino acid sequence to SEQ ID NO: 2.
(b) determining the amino acid sequence of the enolase protein of the whitefly and comparing the amino acid sequence to SEQ ID NO: 4.
(c) determining the amino acid sequence of the fructose bisphosphate protein of the whitefly and comparing the amino acid sequence to SEQ ID NO: 6.

(d) determining the amino acid sequence of the triose phosphate protein of the whitefly and comparing the amino acid sequence to SEQ ID NO: 8.

Preferably, the method comprises investigating a nucleotide sequence or corresponding amino acid sequence of at least one of:
(a) the hsp90 gene of the whitefly,
(b) the enolase gene of the whitefly,
(c) the fructose bisphosphate aldolase gene of the whitefly,
(d) the triose phosphate isomerase gene of the whitefly, or
(e) the vitellogenin gene of the whitefly.

Advantageously, the nucleotide sequence of the hsp90 gene that is investigated is one or more of the 111, 129, 402, 408, 420, 429, 444, 480, 621, 645, 646, 669, 708, 729, 741, 747, 771, 832, 843, 948, 963, 999, 1056, 1092, 1101, 1131, 1134, 1164, 1185, 1189, 1269, 1292, 1368, 1381, 1434, 1484, 1488, 1542, 1578, 1614, 1635, 1647, 1707, 1716, 1728, 1733, 1760, 1770, 1776, 1803, 1822, 1863, 1903, 1943, 1947, 1986, 2019, 2043, 2046, 2049, 2061, 2094, 2112 and 2157 positions of SEQ ID NO: 1.

In one embodiment, a B. tabaci biotype Asia 1 hsp90 gene is identified by the presence of nucleotide T at the 111, 420, 444, 708, 729, 948, 963, 1189, 1647, 1803, 1986 or 2049 position of SEQ ID NO: 1, or the presence of an equivalent genetic marker of 111T, 420T, 444T, 708T, 729T, 948T, 963T, 1189T, 1647T, 1803T, 1822T, 1986T or 2049T of SEQ ID NO: 1.

Suitably, an hsp90 nucleotide sequence is investigated using an oligonucleotide that hybridises with SEQ ID NO: 1.

Preferably, the amino acid sequence that is investigated is one or more of the 224, 439, 503, 586, 595 and 656 positions of SEQ ID NO: 2.

Suitably, a biotype Asia 1 hsp90 polypeptide is identified by the presence of glutamic acid at the 224 position of SEQ ID NO: 2 combined with phenylalanine at the 503 position of SEQ ID NO: 2 and asparagine the 586 position of SEQ ID NO: 2.

According to another aspect of the present invention, there is provided a kit for identifying B. tabaci biotype Asia 1 comprising at least one of:
(a) means for detecting nucleotide T at the 111, 420, 444, 708, 729, 948, 963, 1189, 1647, 1803, 1822, 1986 or 2049 position of SEQ ID NO: 1
(b) means for detecting nucleotide A at the 1185, 1707, 1822 or 1903 position of SEQ ID NO: 1
(c) means for detecting nucleotide C at the 129 or 741 position of SEQ ID NO: 1
(d) means for detecting the presence of a protein derived from the polypeptide sequence of SEQ ID NO: 2, SEQ ID NO: 4, SEQ ID NO: 6 or SEQ ID NO: 8.

Preferably, the means of (a)-(c) is one or more oligonucleotide.

Advantageously, the means of (d) is an antibody.

In another aspect, the present invention provides an antibody or fragment thereof which specifically binds to a polypeptide, the polypeptide comprising: at least 10 contiguous amino acids from the amino acid sequence of SEQ ID NO: 2, SEQ ID NO: 4, SEQ ID NO: 6 or SEQ ID NO: 8, or an immunogenic fragment of the amino acid sequence of SEQ ID NO: 2, SEQ ID NO: 4, SEQ ID NO: 6 or SEQ ID NO: 8.

In a further aspect, the present invention provide an isolated polypeptide selected from:
(a) an amino acid sequence comprising SEQ ID NO: 2, SEQ ID NO: 4, SEQ ID NO: 6 or SEQ ID NO: 8
(b) an immunogenic fragment of the amino acid sequence of (a)
(c) an amino acid sequence comprising at least 10 contiguous amino acids from SEQ ID NO: 2, SEQ ID NO: 4, SEQ ID NO: 6 or SEQ ID NO: 8

In a further aspect, the present invention provides an isolated polynucleotide as described above:
(a) a polynucleotide which encodes a polypeptide as described above
(b) a polynucleotide comprising SEQ ID NO: 1, SEQ ID NO: 3, SEQ ID NO: 5, SEQ ID NO: 7 or SEQ ID NO: 9
(c) a polynucleotide comprising a sequence which is complementary to (a) or (b)
(d) a polynucleotide which hybridises under stringent conditions to any one of (a) to (c)
(e) a polynucleotide comprising at least 10 contiguous nucleotides from any one of (a) to (d)

The present invention further provides an oligonucleotide or compliment thereof that hybridises to a region of SEQ ID NO: 1, SEQ ID NO: 3, SEQ ID NO: 5, SEQ ID NO: 7 or SEQ ID NO: 9.

Preferably, the oligonucleotide hybridises to a region spanning at least one of the 111, 420, 444, 708, 729, 948, 963, 1189, 1647, 1803, 1822, 1986 or 2049 position of SEQ ID NO: 1

More preferably, the oligonucleotide comprises 10-20 nucleotides

In a further aspect, the present invention provides a composition comprising a polypeptide or an antibody as described above.

In another aspect, the present invention provides a hybridisation probe, peptide nucleic acid, membrane, microarray or ribozyme comprising a polynucleotide as described above.

Another aspect of the present invention is the use of an agent that recognises the polypeptide of one or more of SEQ ID NO: 2, SEQ ID NO: 4, SEQ ID NO: 6 or SEQ ID NO: 8 as an agrochemical.

In a final aspect, the present invention provides a method of screening a library of molecules or compounds to identify a ligand which binds the polypeptide of one or more of SEQ ID NO: 2, SEQ ID NO: 4, SEQ ID NO: 6 or SEQ ID NO: 8.

Preferably, the ligand is an agrochemical.

The above and other aspects of the present invention will now be described in further detail, by way of example only, with reference to the accompanying figures and examples, in which:

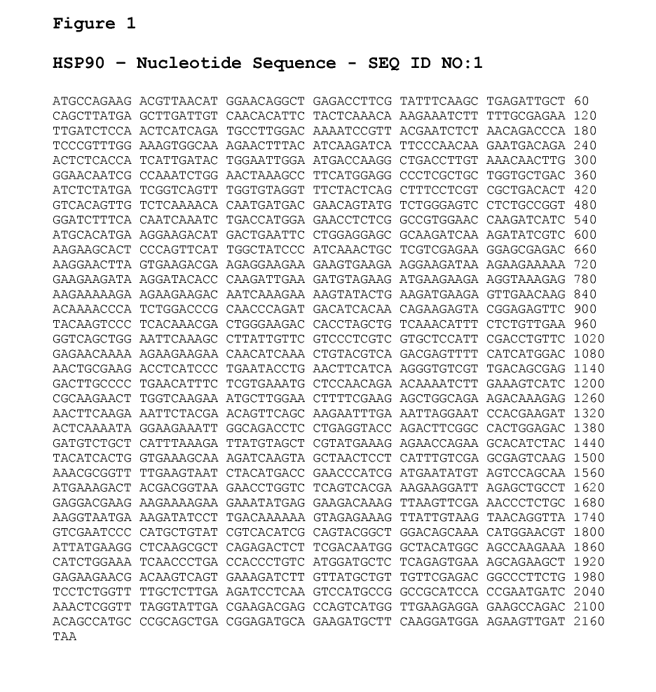

FIG. 1 is a cDNA nucleotide sequence of B. tabaci Asia 1 biotype hsp90 (coding sequence)—SEQ ID NO: 1;

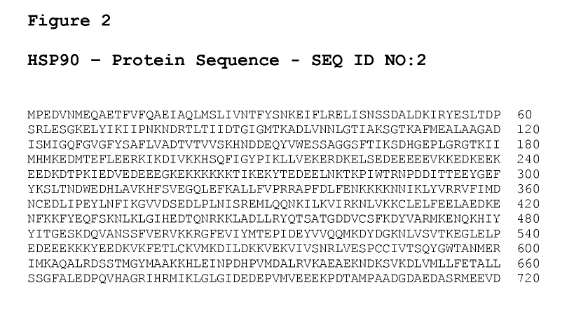

FIG. 2 is an amino acid sequence of B. tabaci Asia 1 biotype hsp90—SEQ ID NO: 2;

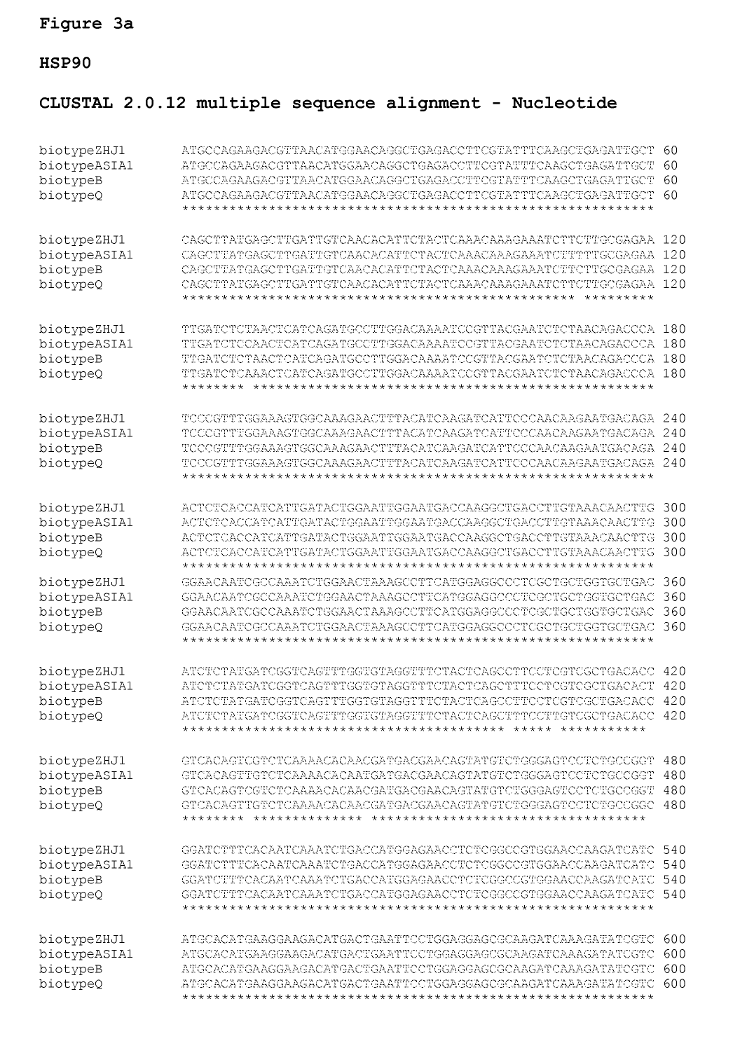

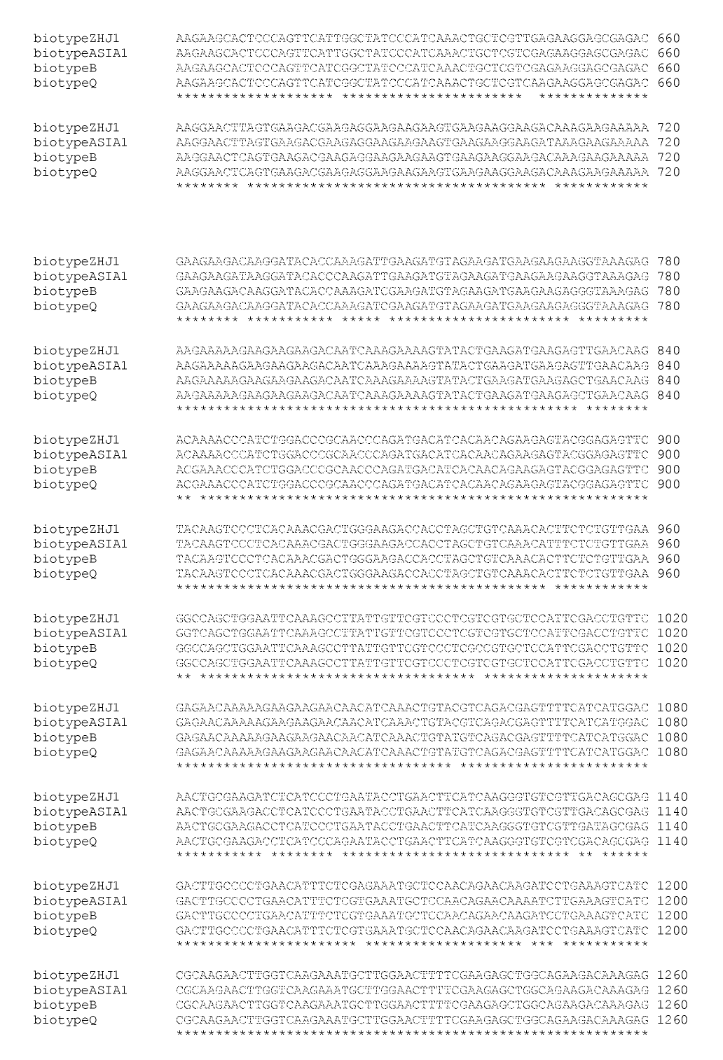

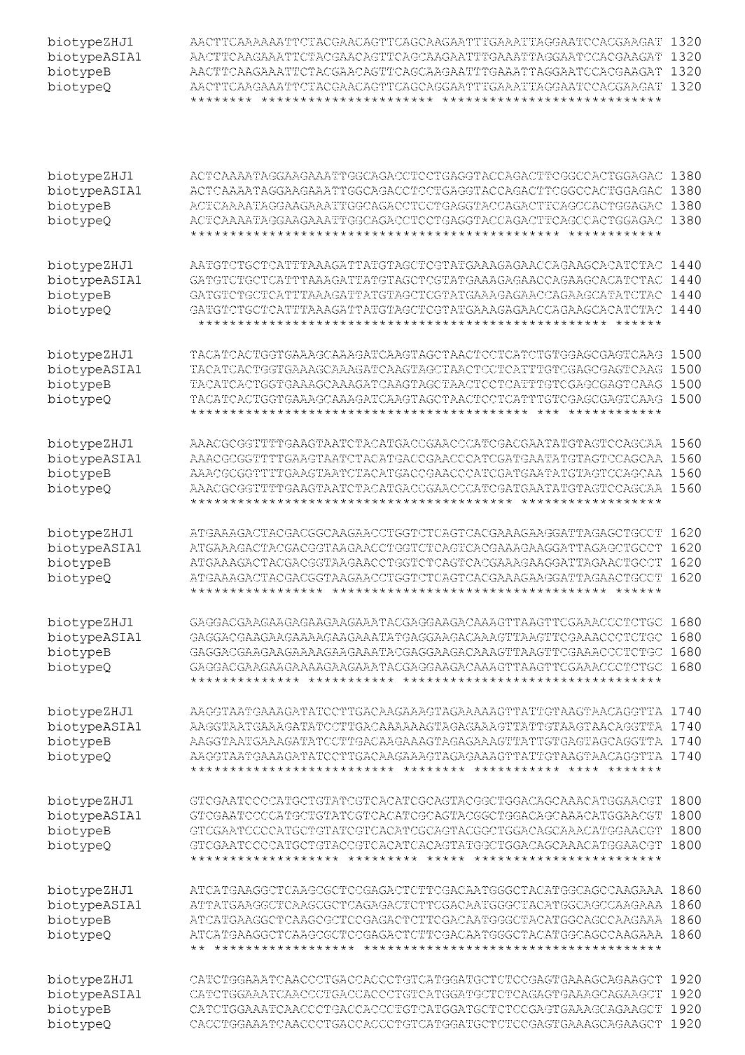

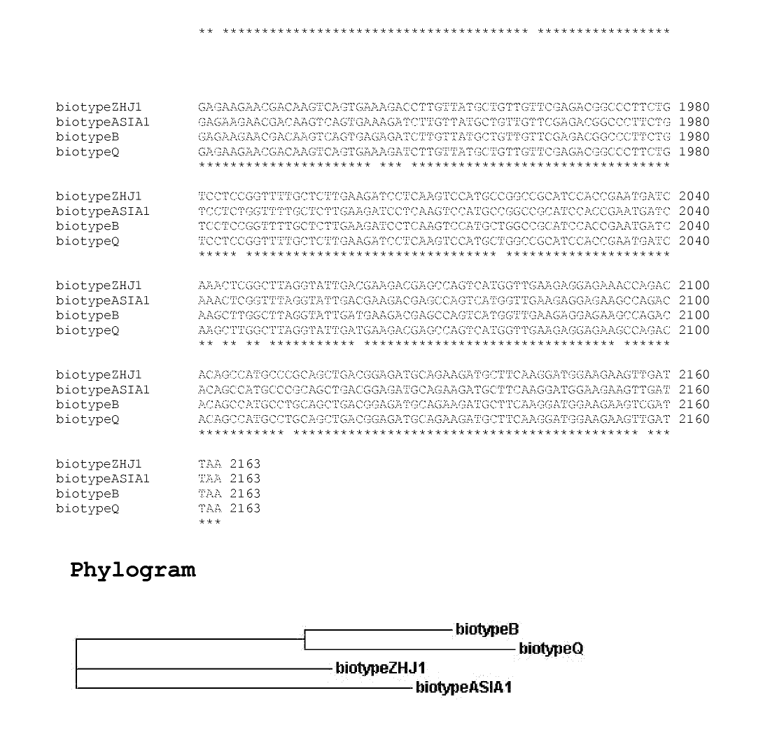

FIG. 3 is an alignment of the nucleotide sequence of FIG. 1 with B. tabaci biotype ZHJ1, biotype B and biotype Q hsp90 sequences—(a) DQ093381.1 biotype B, (b)

DQ093380.1 biotype B, (c) EU934241.1 biotype B;

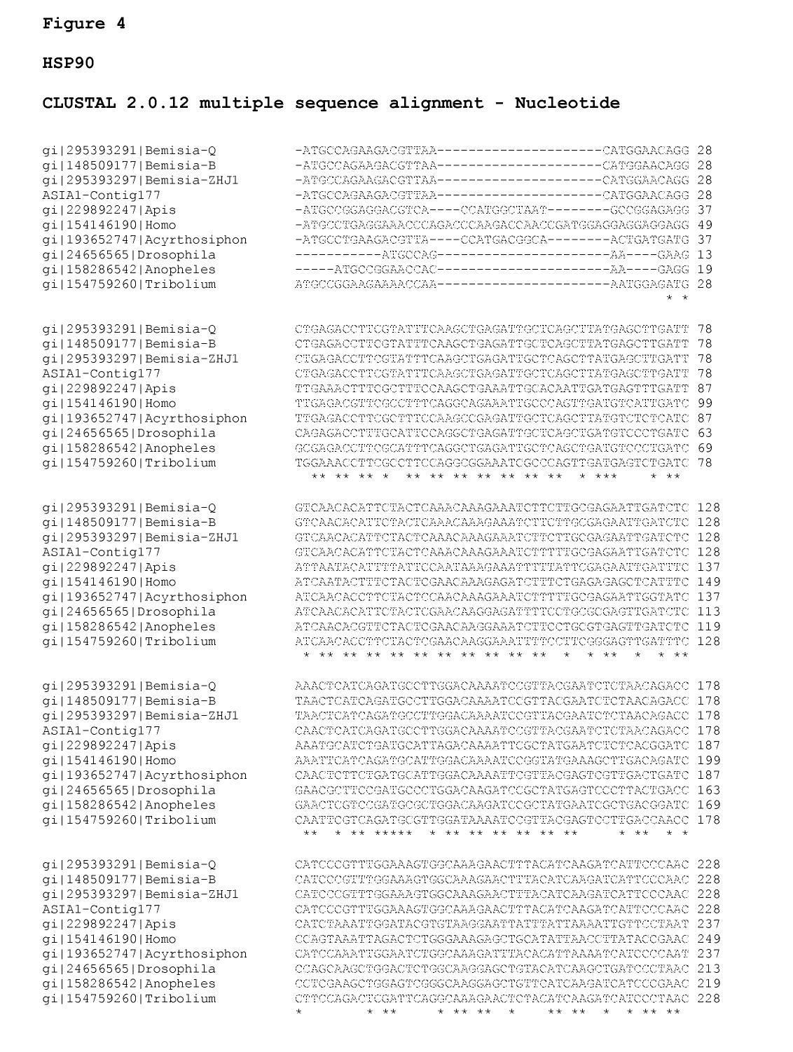

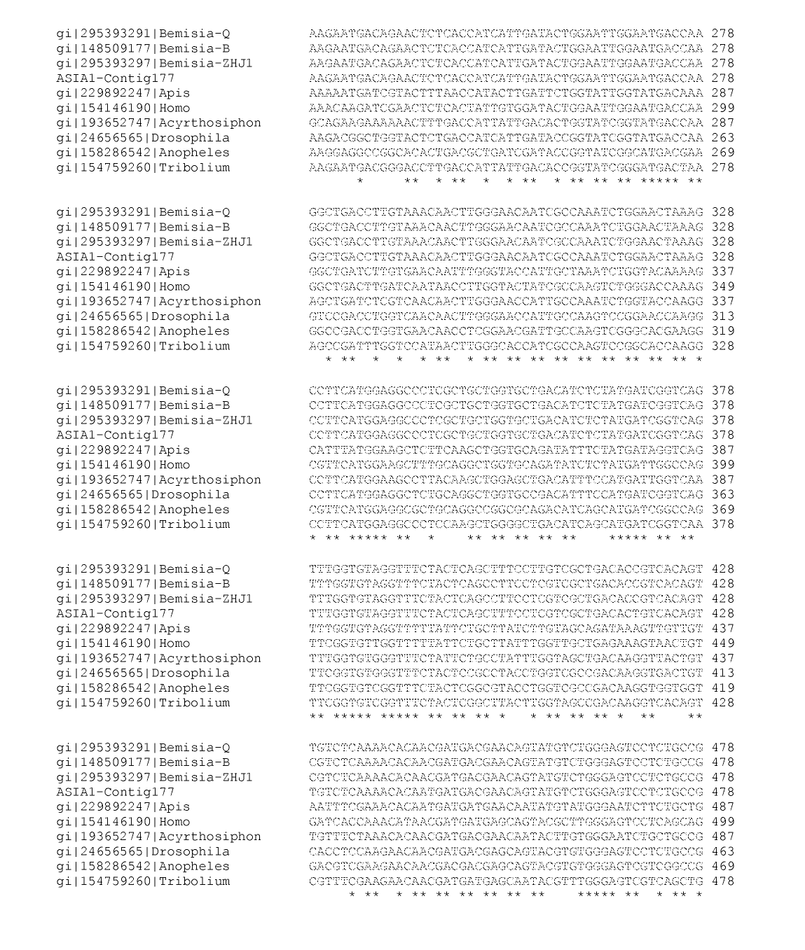

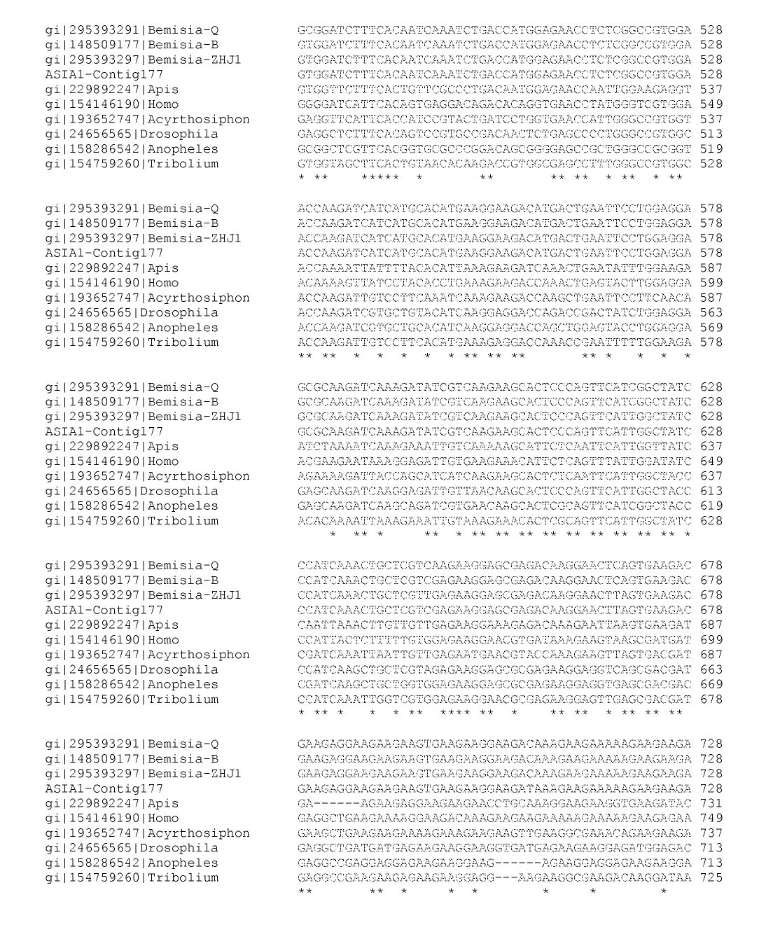

FIG. 4 is the alignment of FIG. 3 including sequences from additional species;

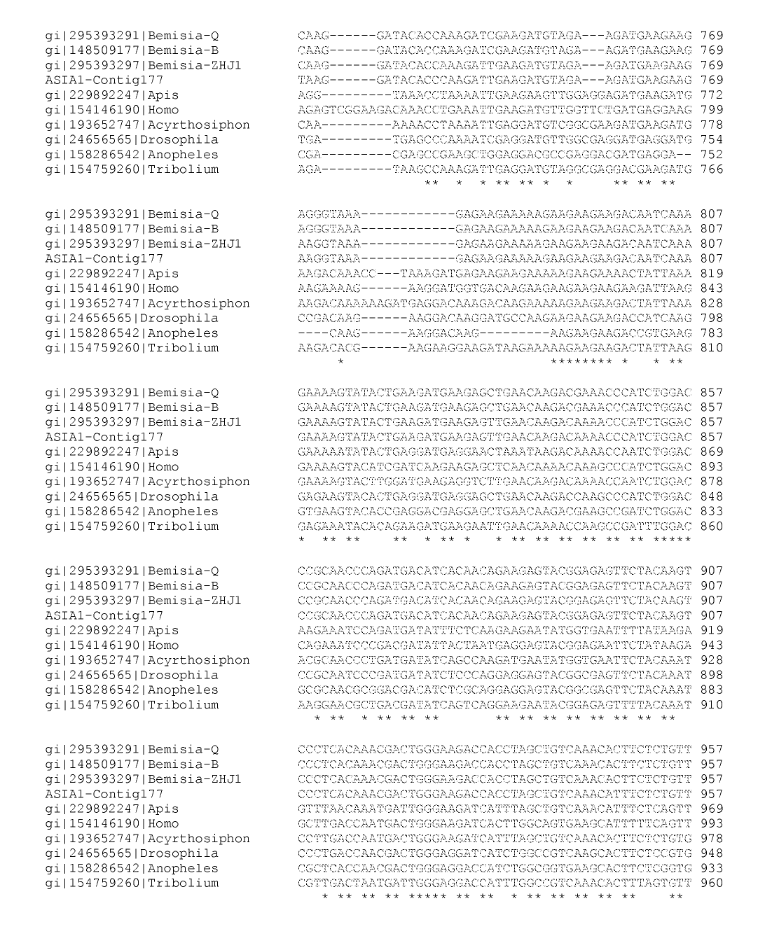

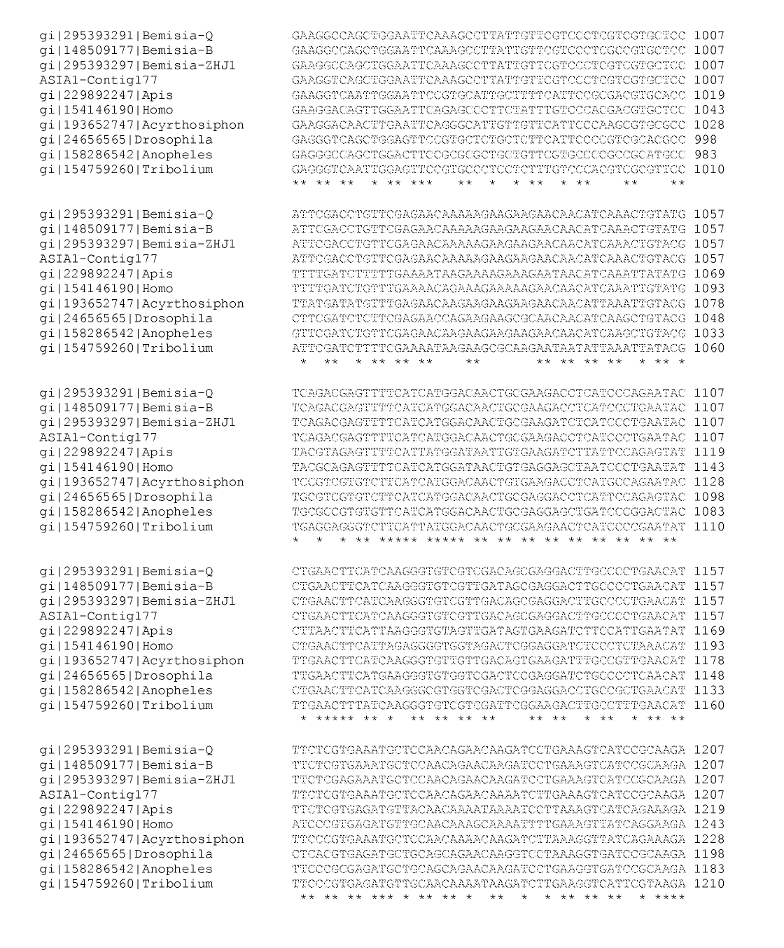

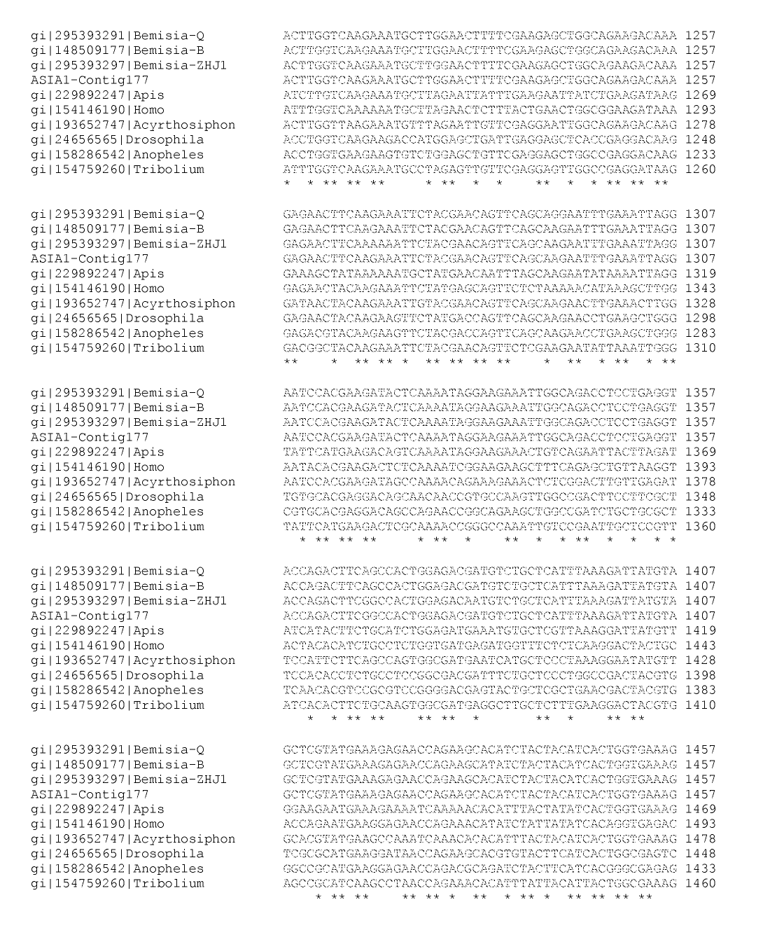

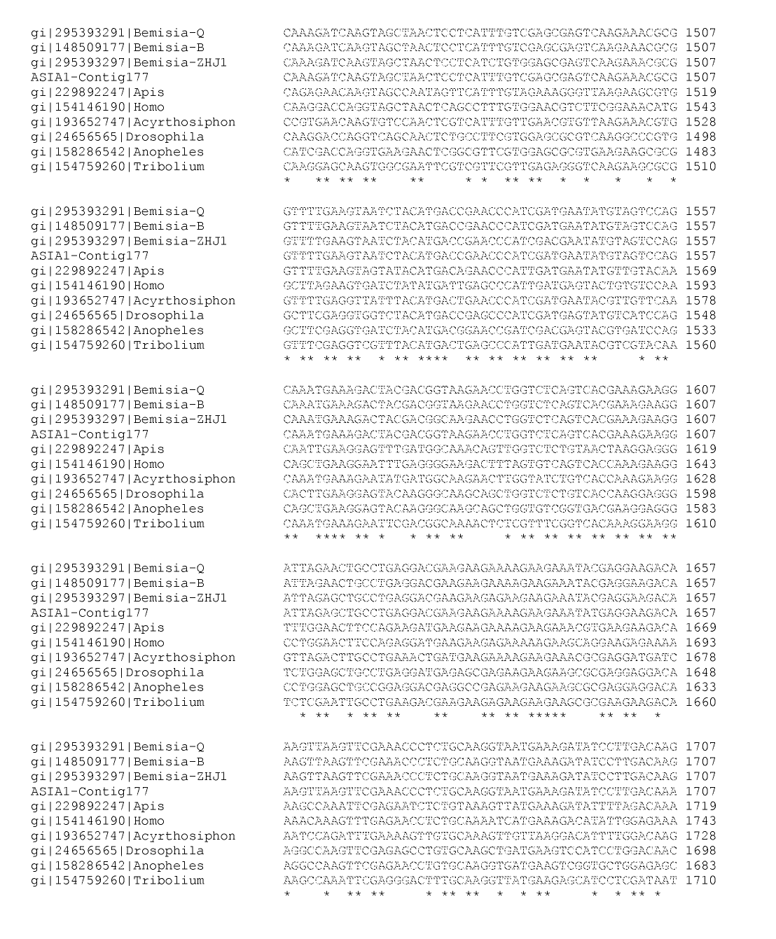

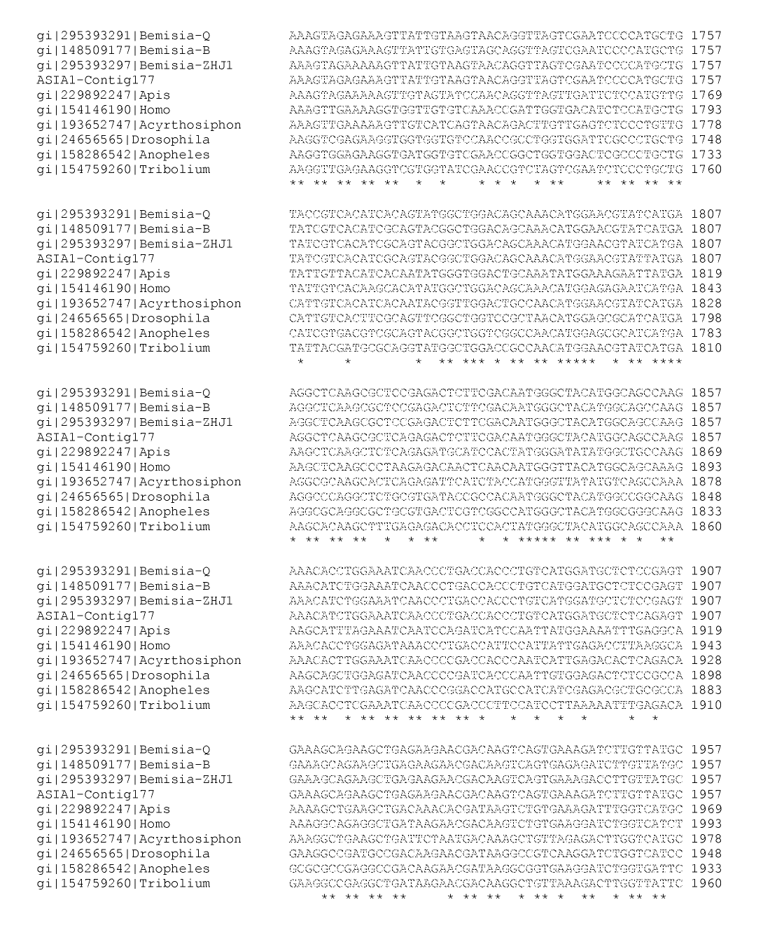

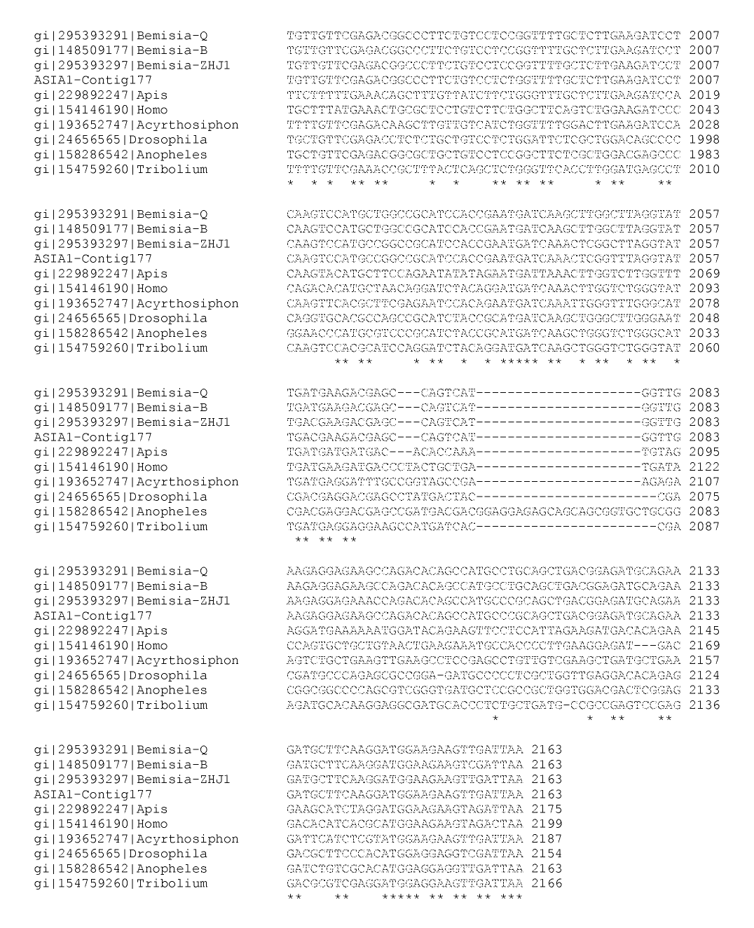

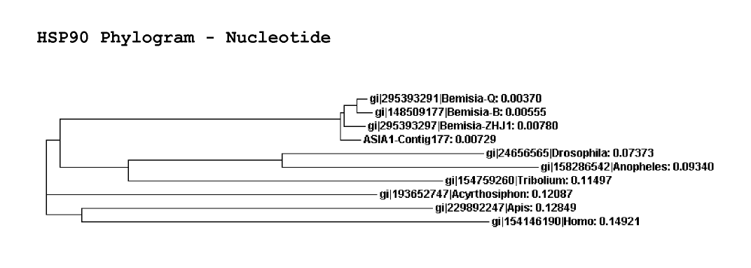

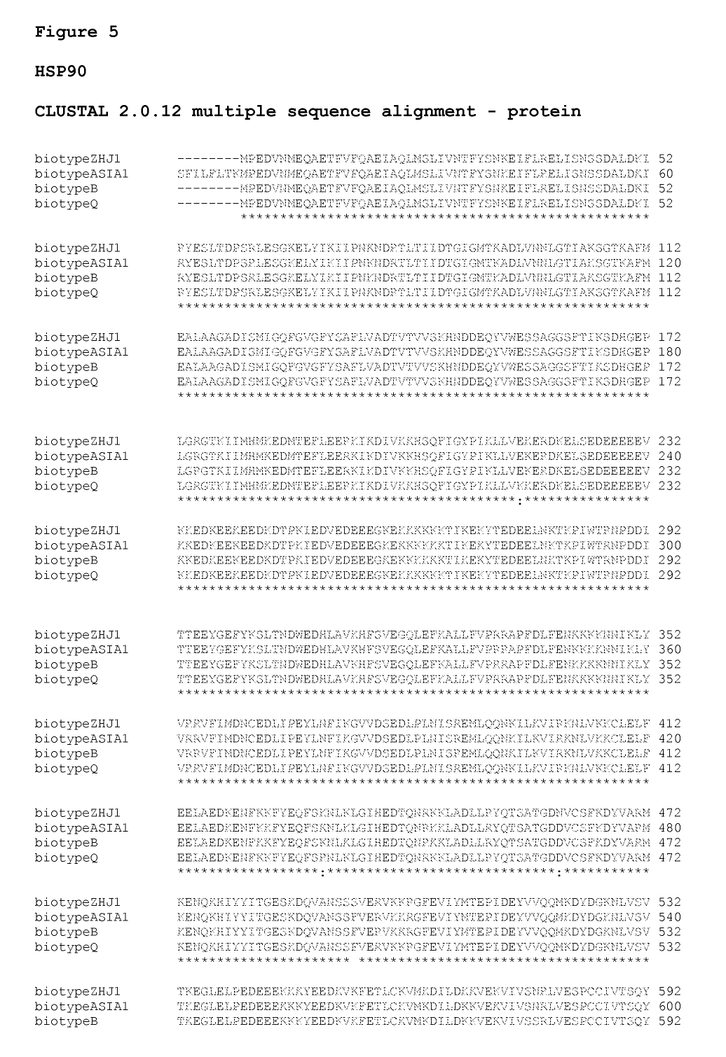

FIG. 5 is an alignment of the amino acid sequence of FIG. 2 with B. tabaci biotype ZHJ1, biotype B and biotype Q hsp90 sequences;

FIG. 6 is the alignment of FIG. 5 including sequences from additional species;

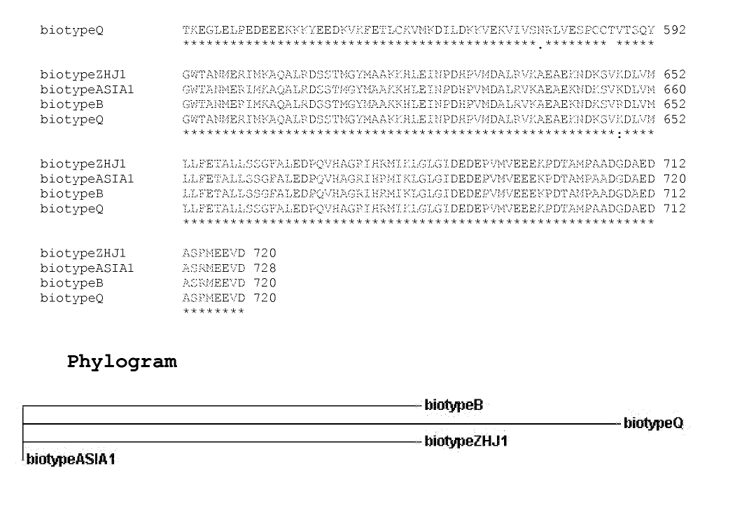

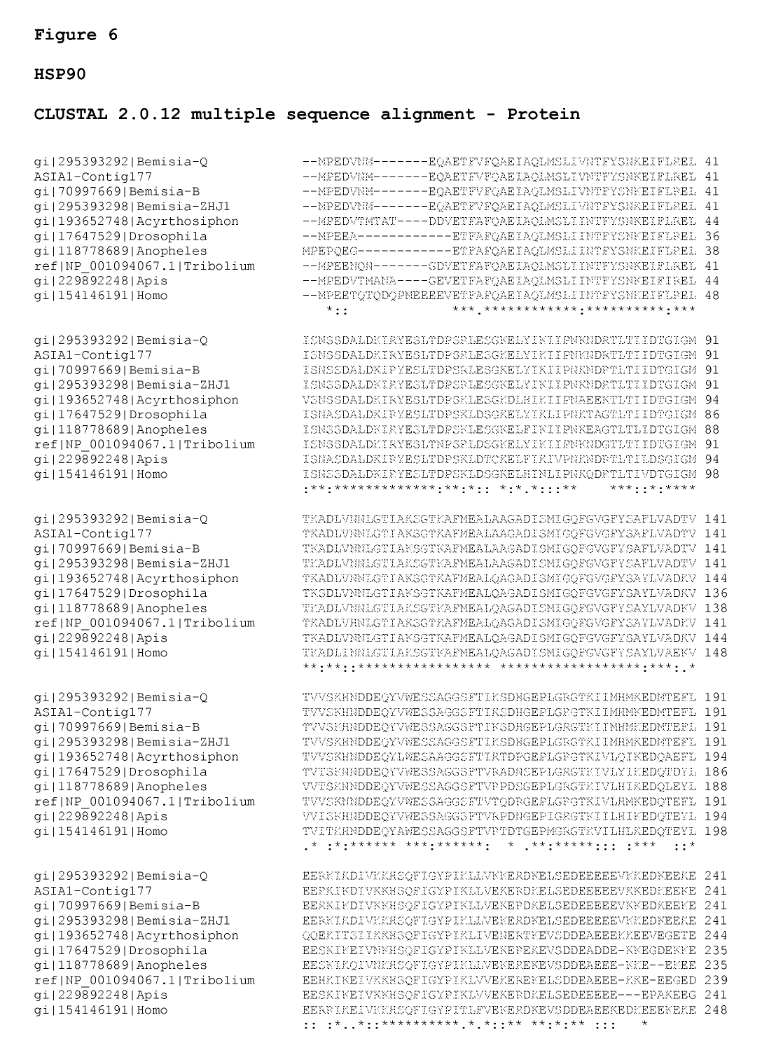

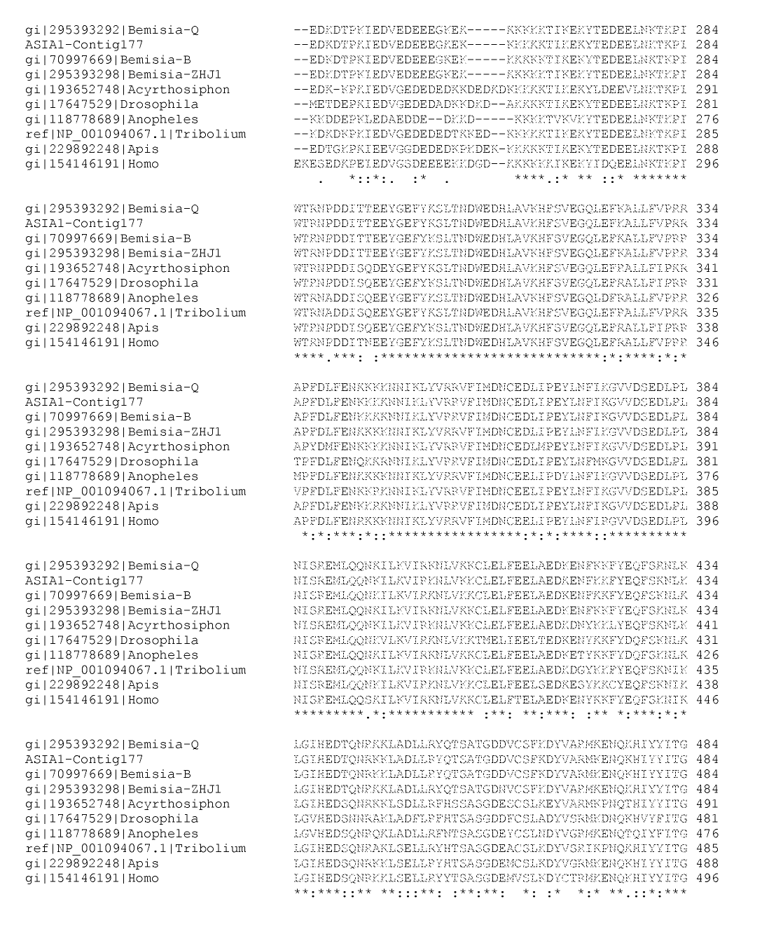

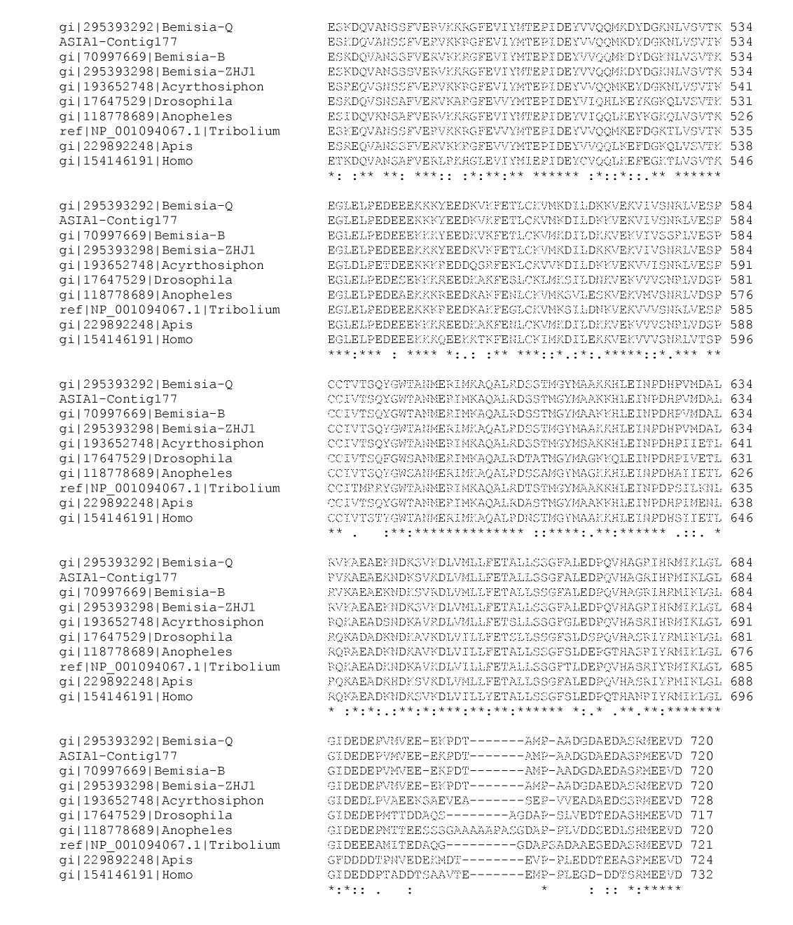

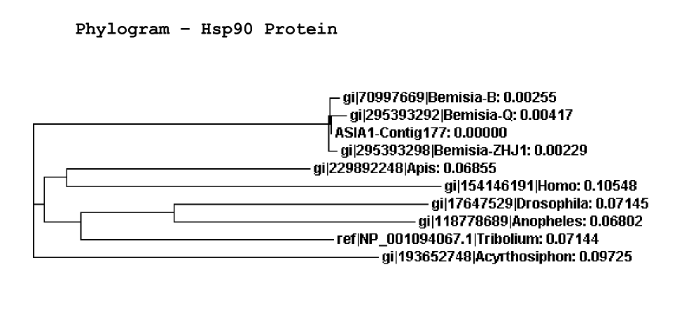

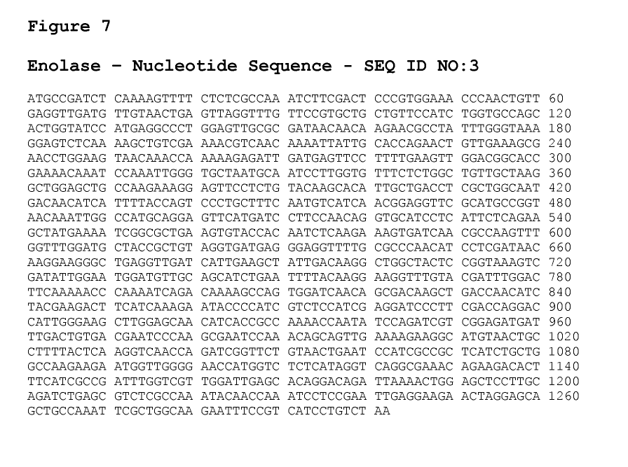

FIG. 7 is a cDNA nucleotide sequence of B. tabaci Asia 1 biotype enolase (coding sequence)—SEQ ID NO: 3;

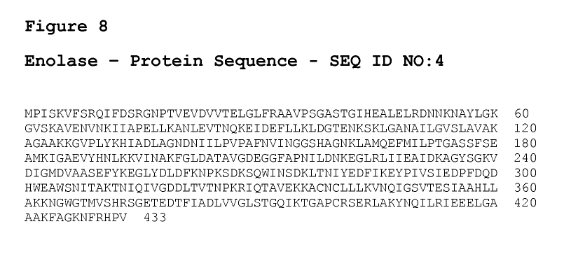

FIG. 8 is an amino acid sequence of B. tabaci Asia 1 biotype enolase—SEQ ID NO: 4

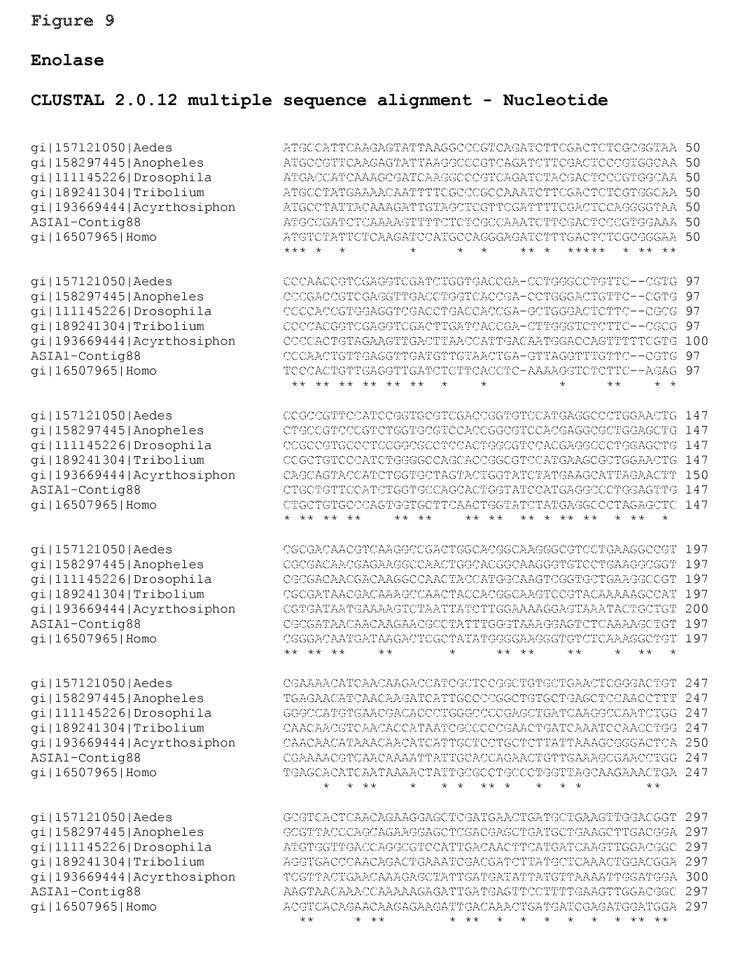

FIG. 9 is an alignment of the nucleotide sequence of FIG. 7 with sequences from other species;

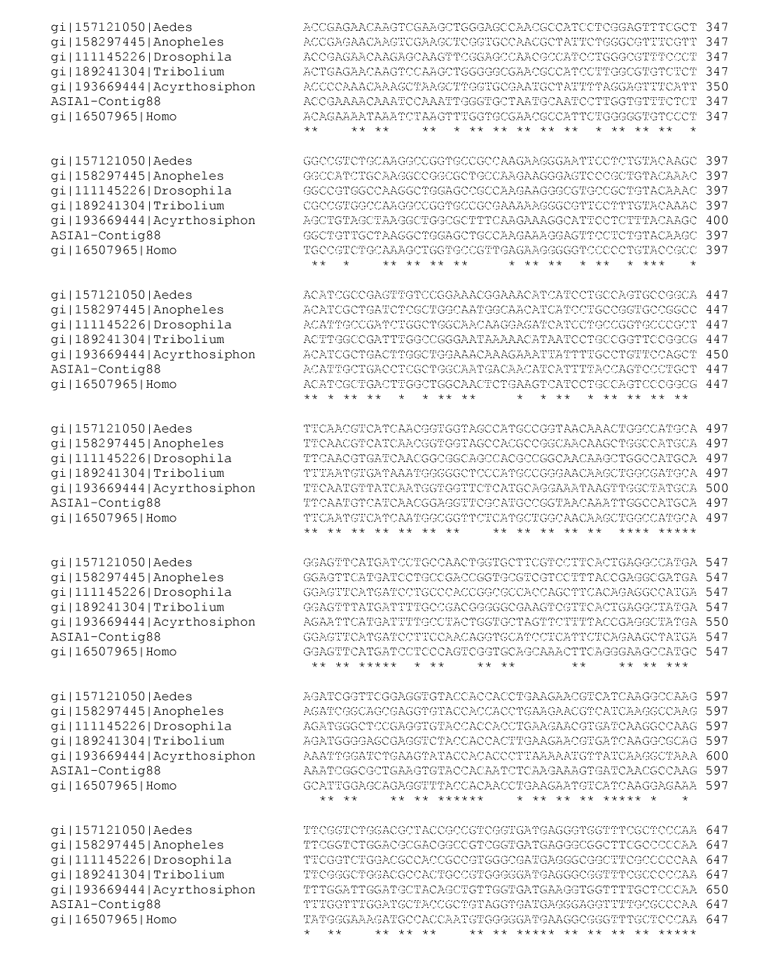

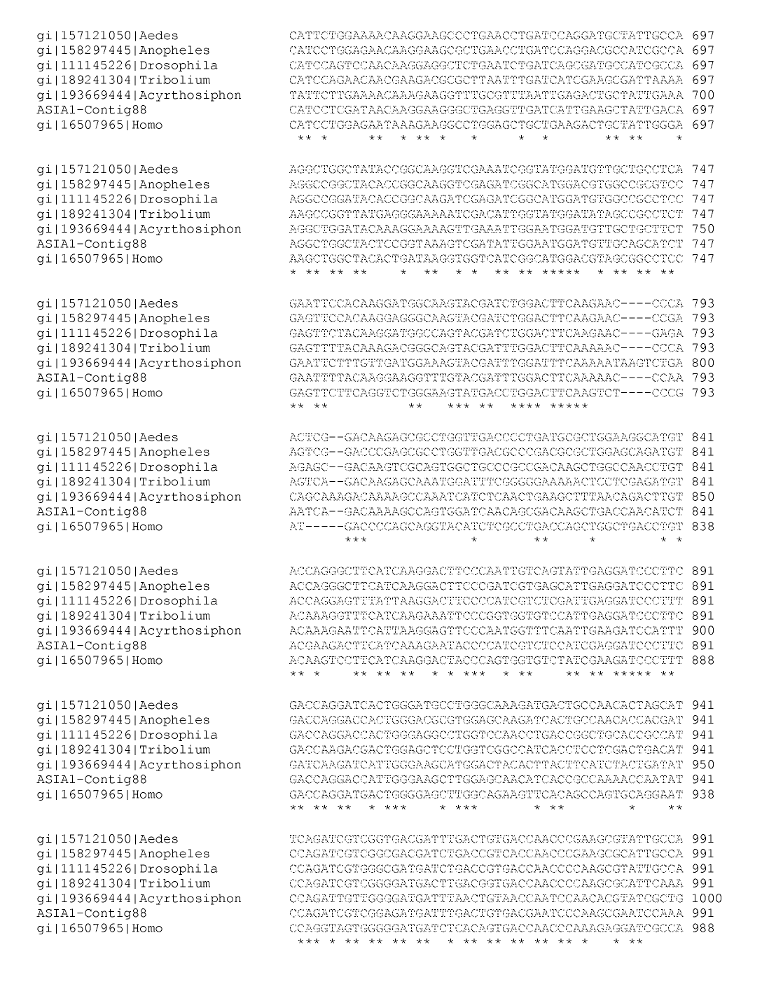

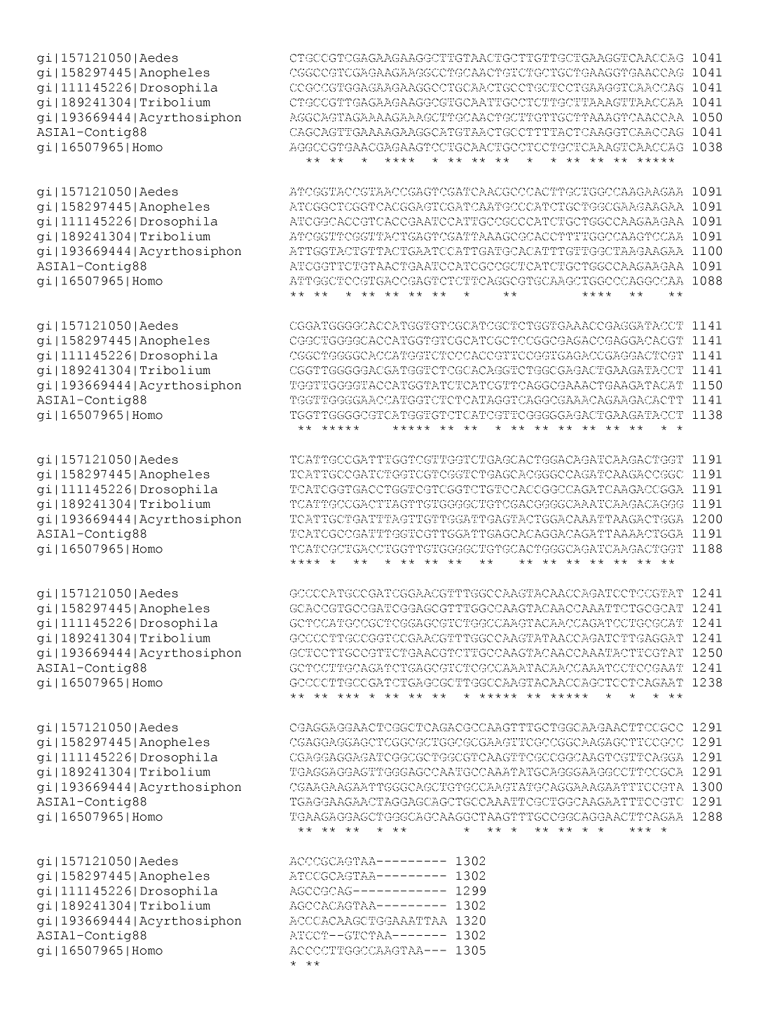

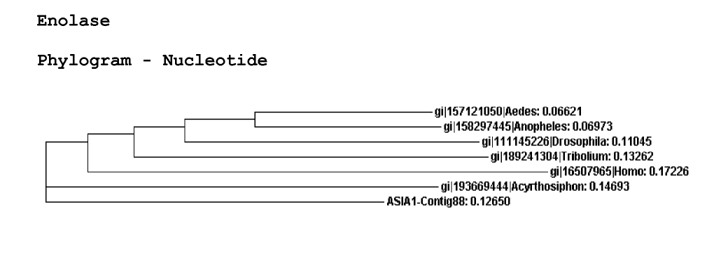

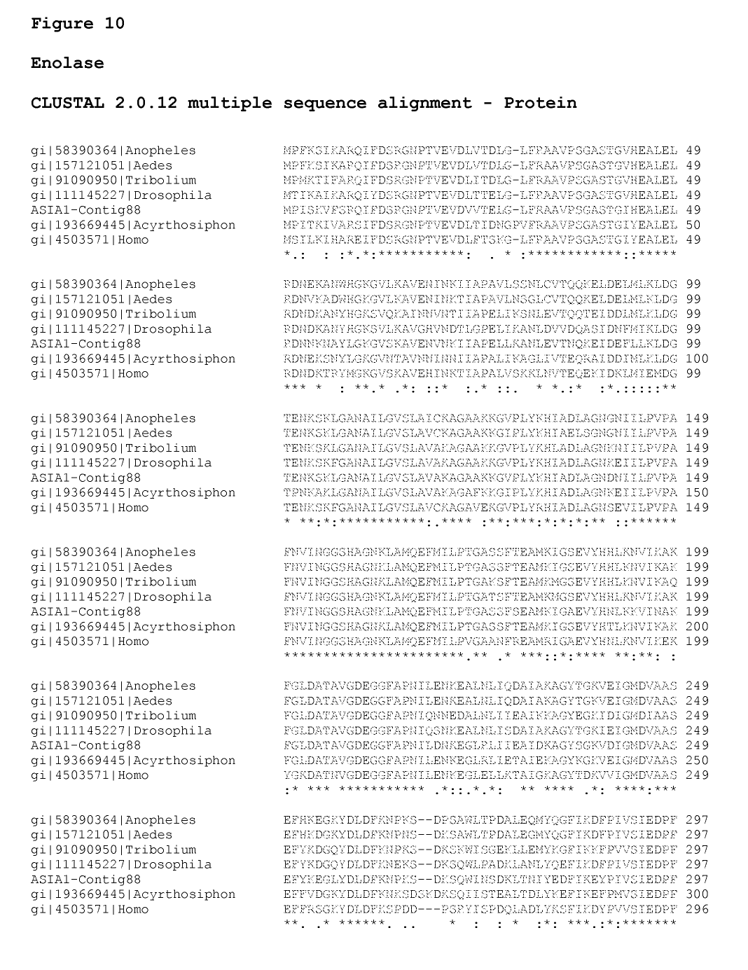

FIG. 10 is an alignment of the amino acid sequence of FIG. 8 with sequences from other species;

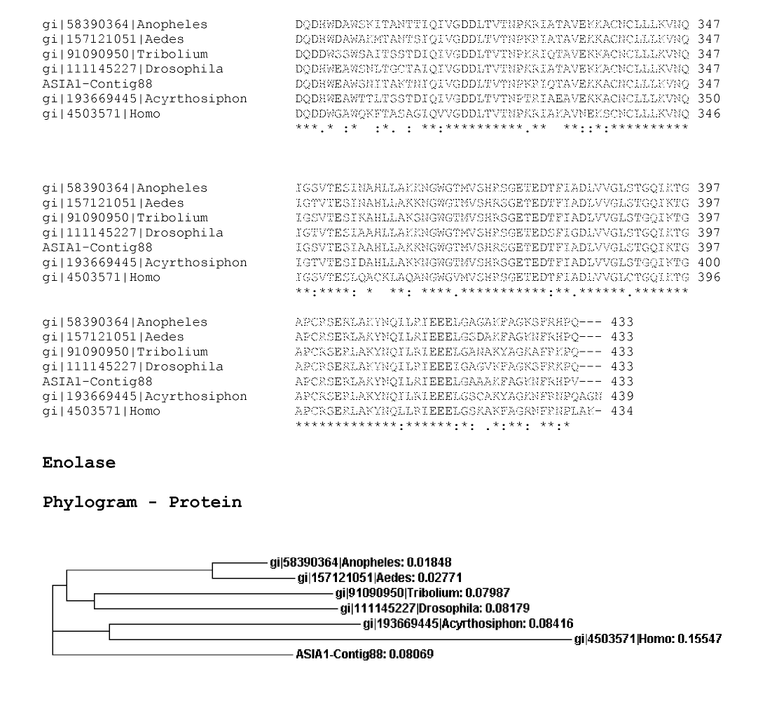

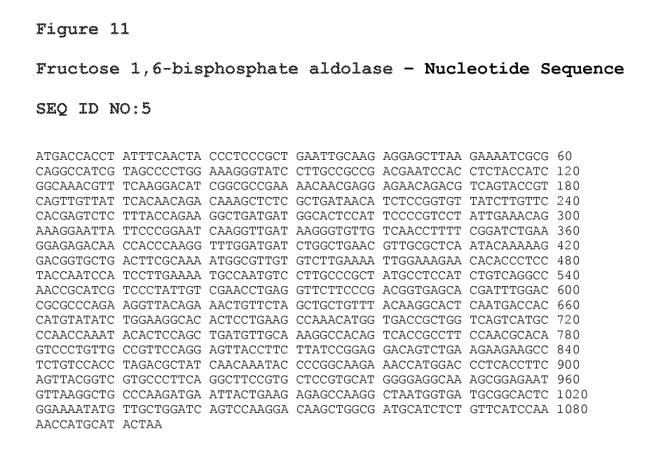

FIG. 11 is a cDNA nucleotide sequence of B. tabaci Asia 1 biotype fructose 1,6-bisphosphate aldolase (coding sequence)—SEQ ID NO: 5;

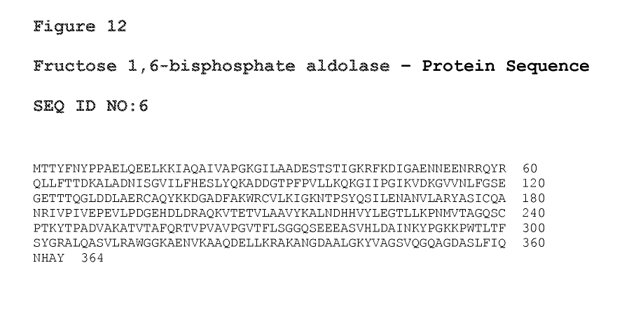

FIG. 12 is an amino acid sequence of B. tabaci Asia 1 biotype 1,6-bisphosphate aldolase—SEQ ID NO: 6;

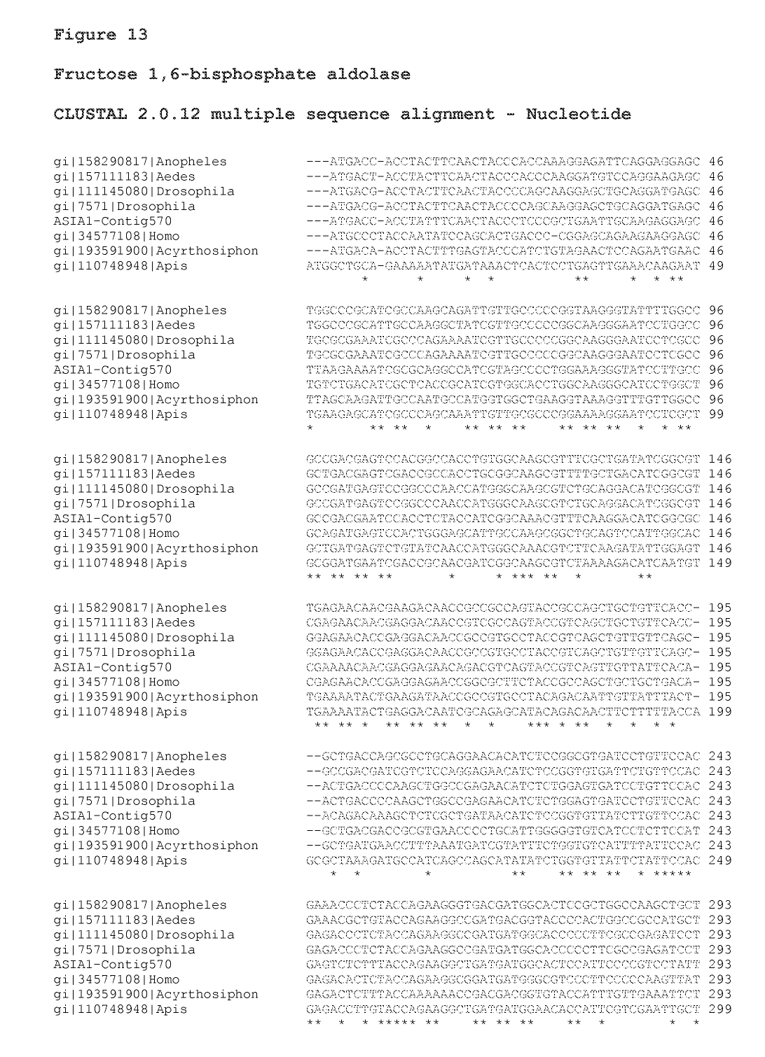

FIG. 13 is an alignment of the nucleotide sequence of FIG. 11 with sequences from other species;

FIG. 14 is an alignment of the amino acid sequence of FIG. 12 with sequences from other species;

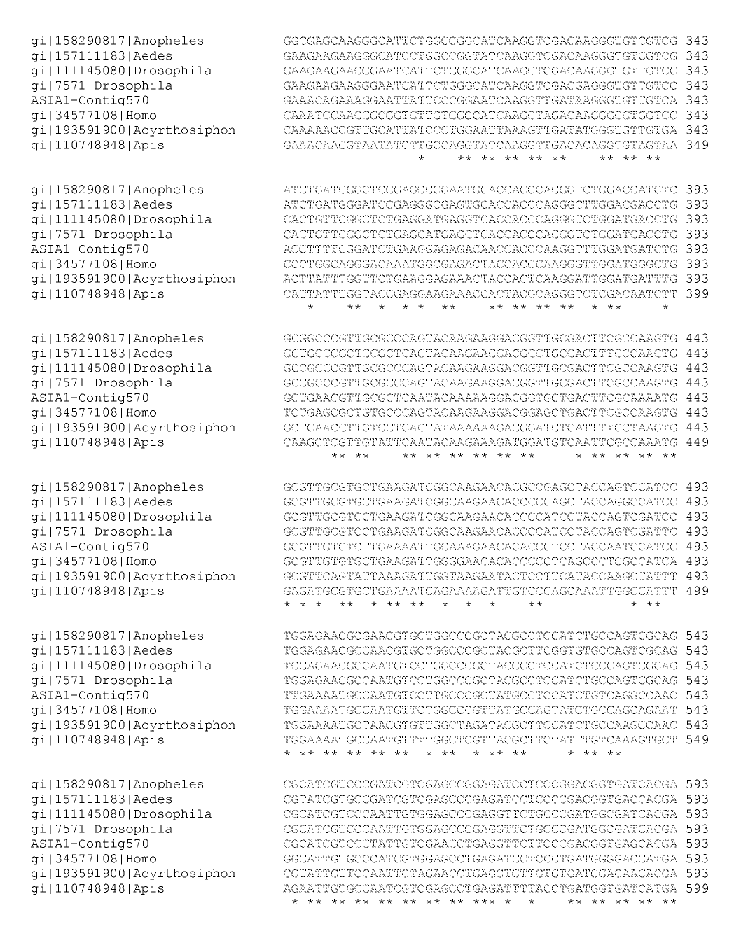

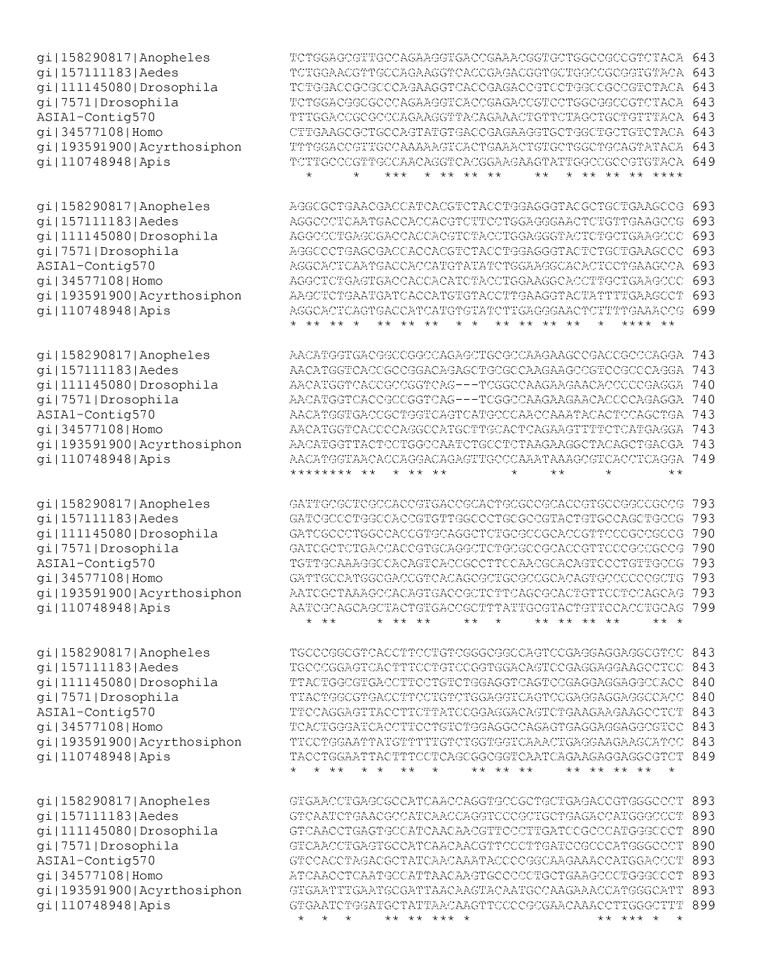

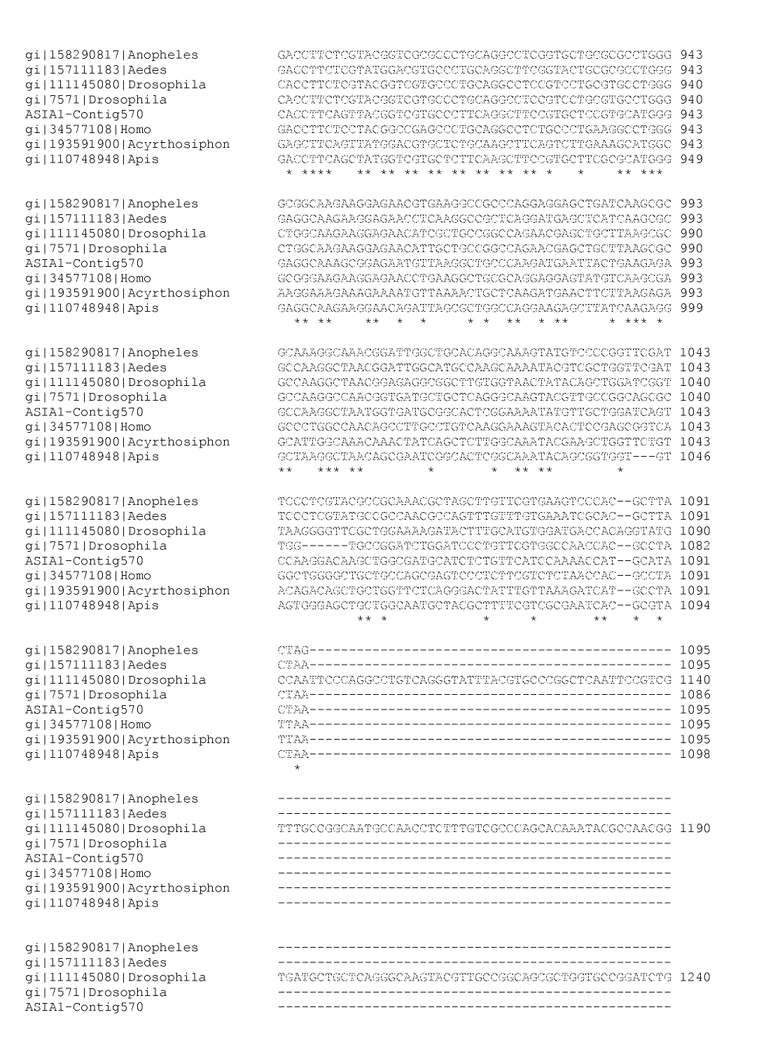

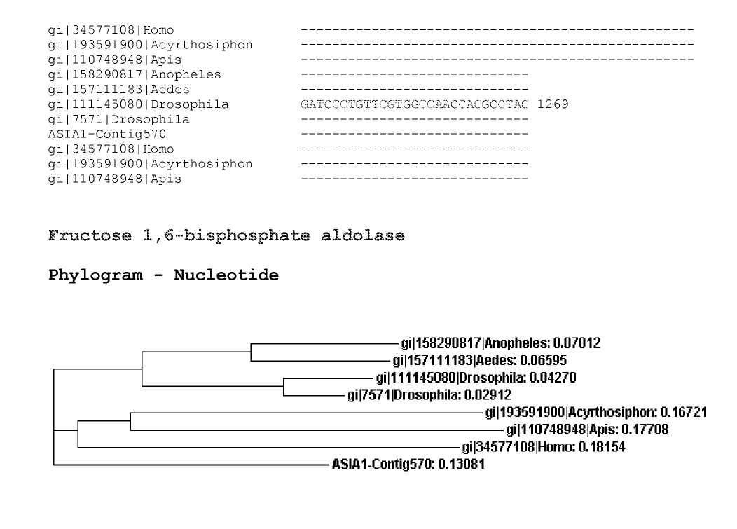

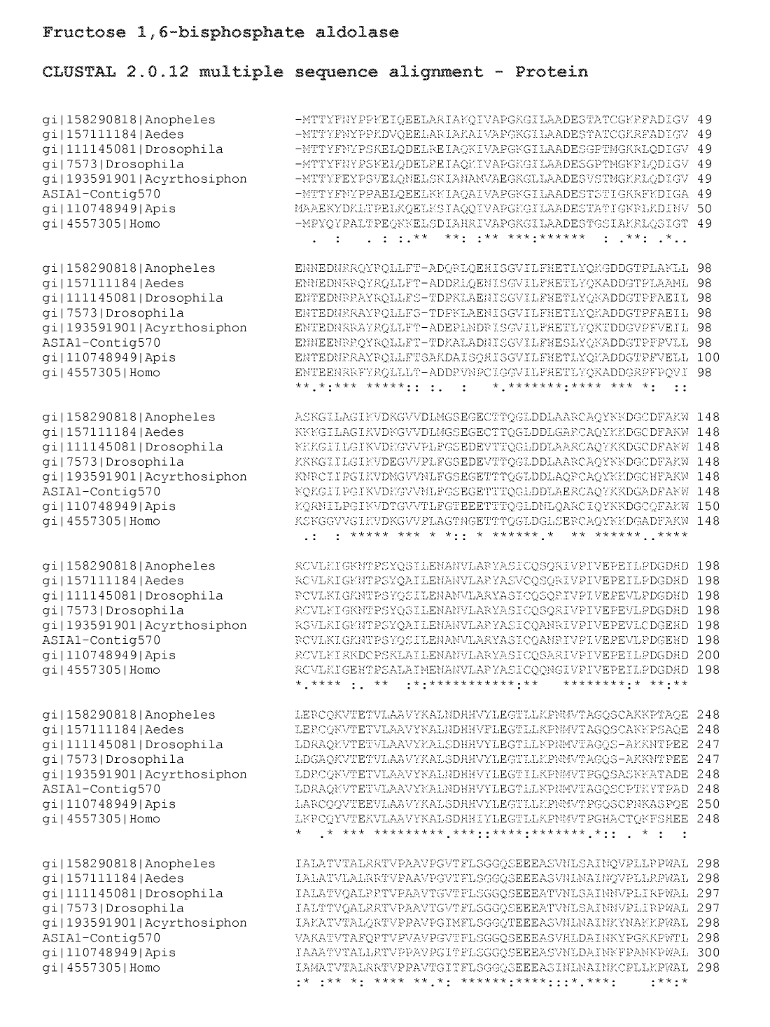

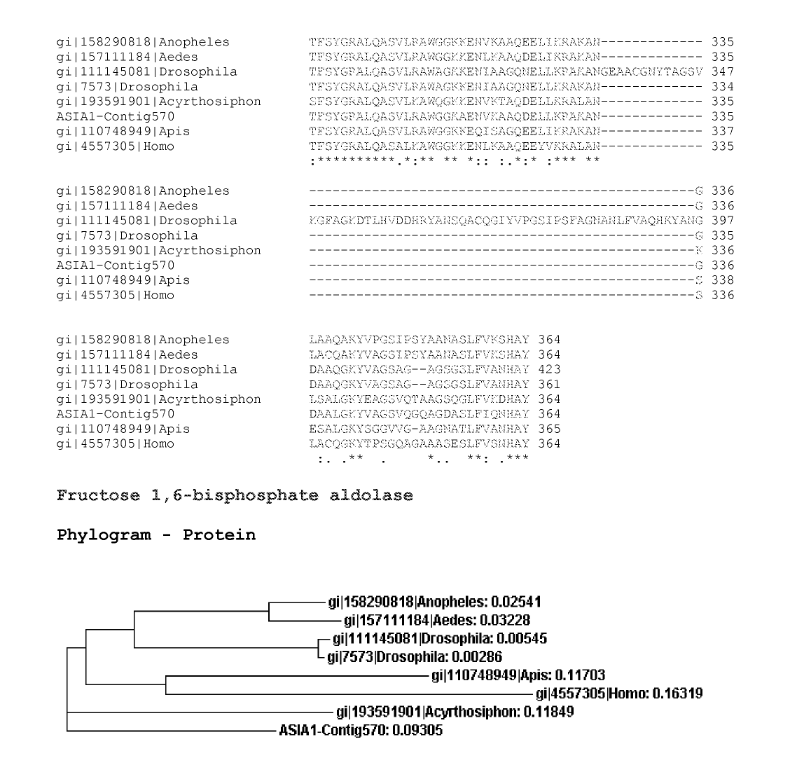

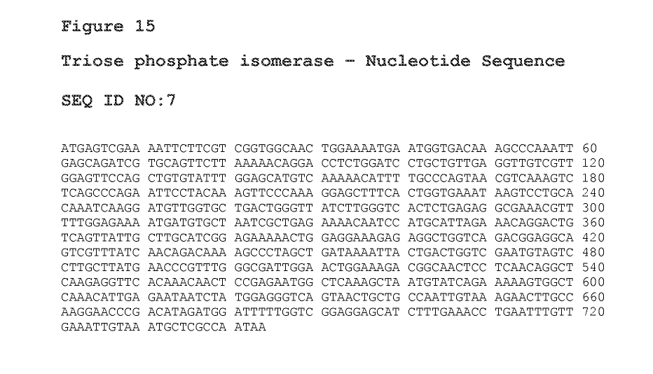

FIG. 15 is a cDNA nucleotide sequence of B. tabaci Asia 1 biotype triose phosphate isomerase (coding sequence)—SEQ ID NO: 7;

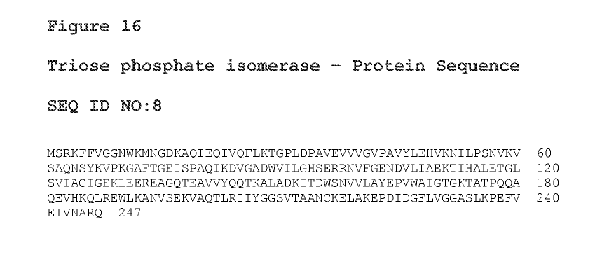

FIG. 16 is an amino acid sequence of B. tabaci Asia 1 biotype triose phosphate isomerase—SEQ ID NO: 8;

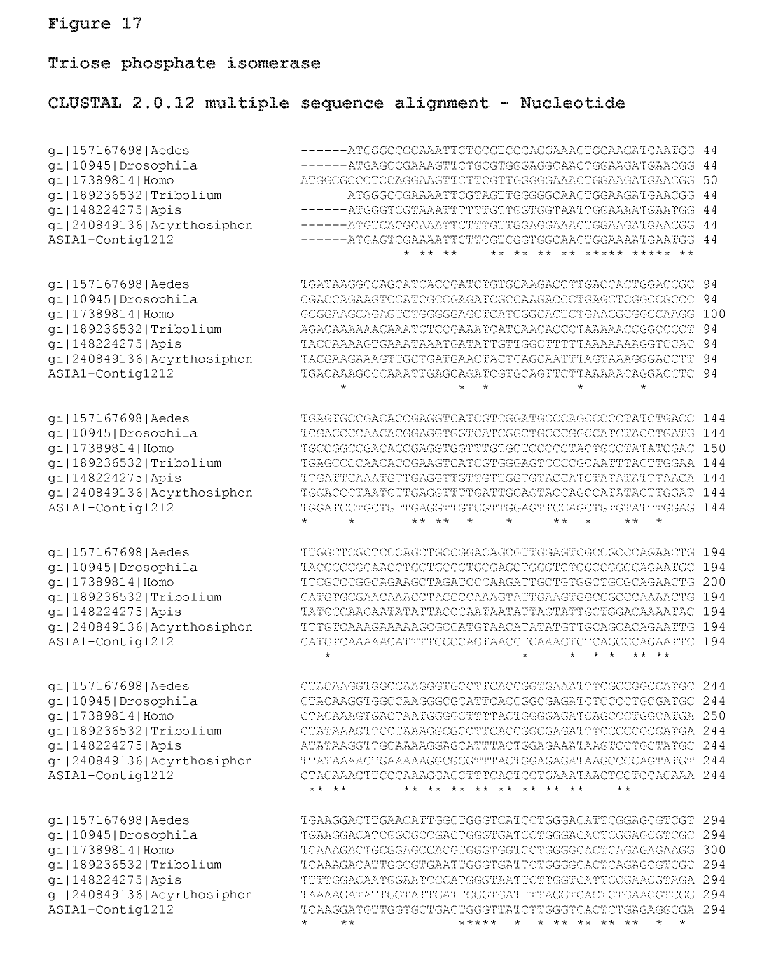

FIG. 17 is an alignment of the nucleotide sequence of FIG. 15 with sequences from other species;

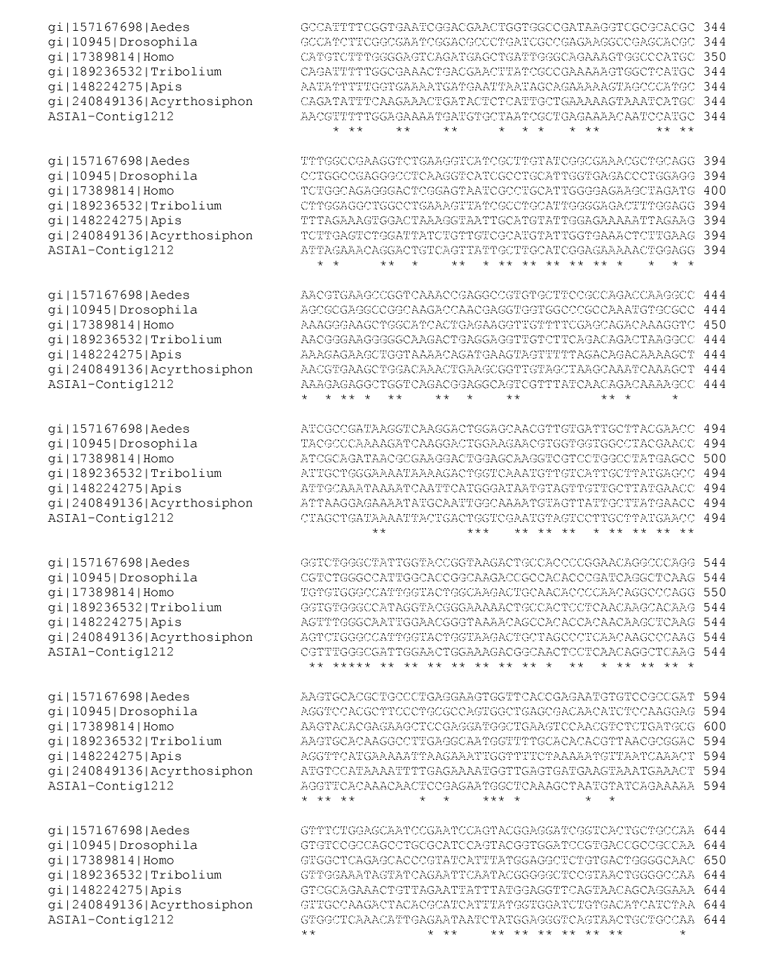

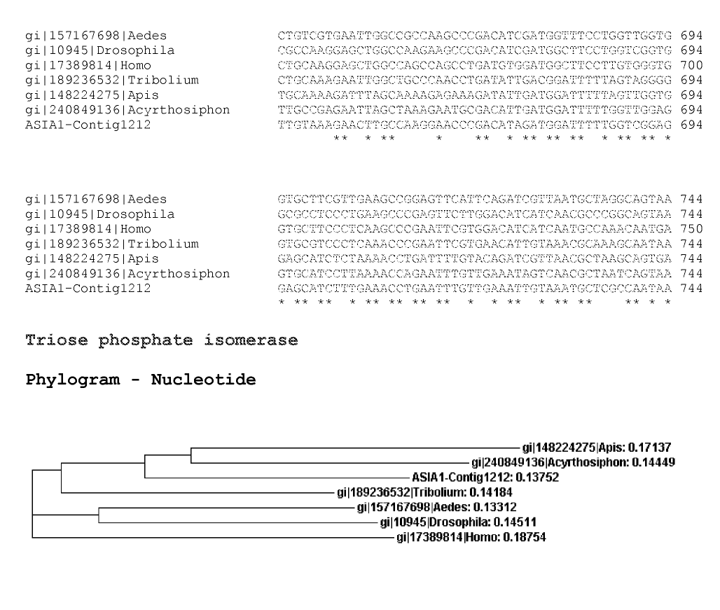

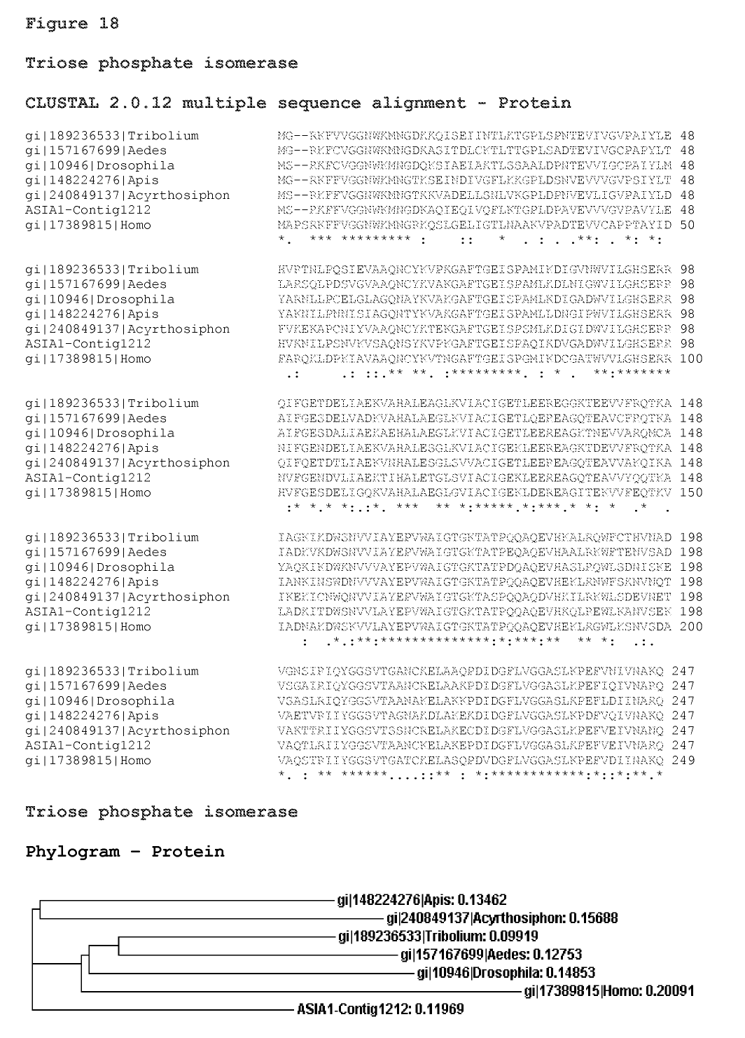

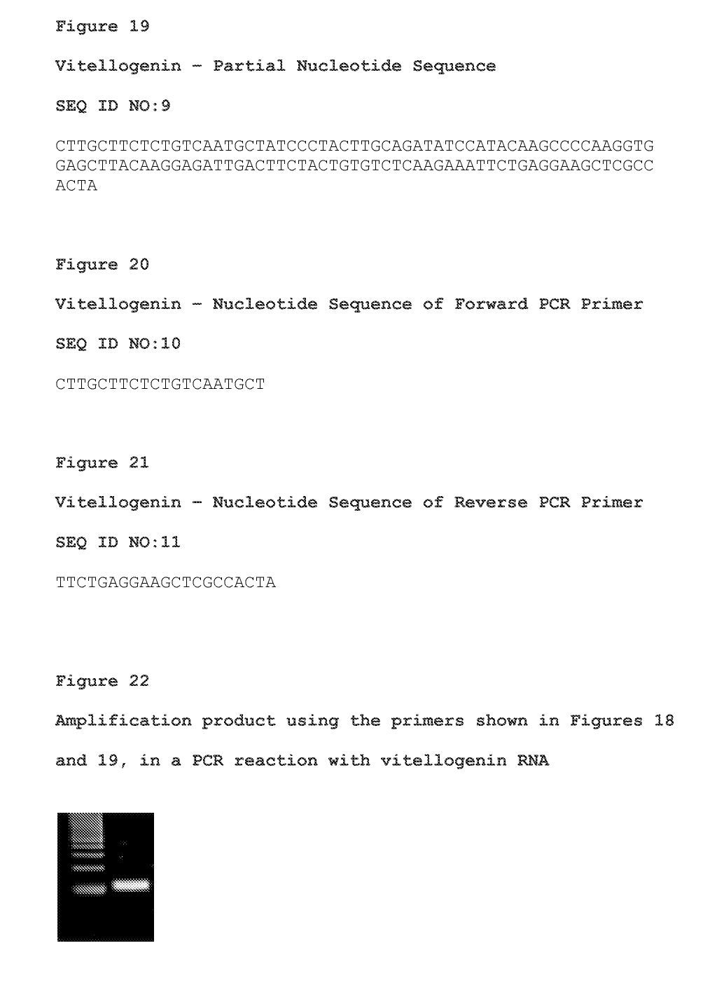

FIG. 18 is an alignment of the amino acid sequence of FIG. 16 with sequences from other species;

FIG. 19 is a partial cDNA nucleotide sequence of a B. tabaci Asia 1 biotype putative vitellogenin (coding sequence)—SEQ ID NO: 9;

FIG. 20 is a nucleotide sequence of a vitellogenin forward PCR primer—SEQ ID NO: 10;

FIG. 21 is a nucleotide sequence of a vitellogenin reverse PCR primer—SEQ ID NO: 11;

FIG. 22 shows a gel comprising an amplification product using the primers of

FIGS. 20 and 21 in a PCR reaction with vitellogenin RNA;

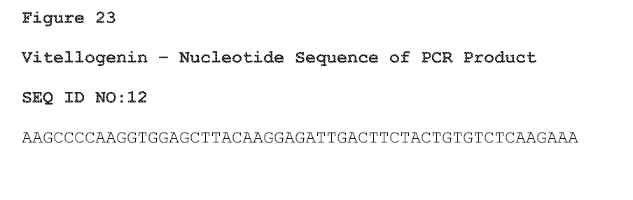

FIG. 23 is a nucleotide sequence of the amplification product of FIG. 22—SEQ ID NO: 12; and

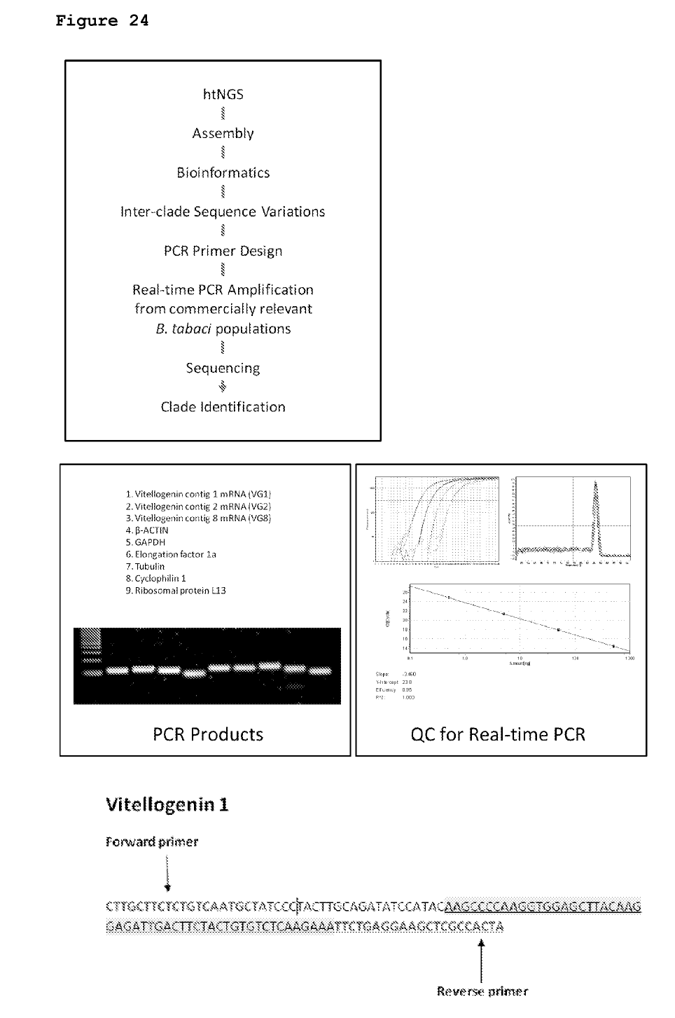

FIG. 24 shows qPCR results using vitellogenin RNA.

Over 3,000,000 genetic segments from biotype Asia 1 whitefly were sequenced using high-throughput next generation DNA sequencing technologies (htNGS). Adult female B. tabaci cDNA libraries were produced using an Evrogen MINT cDNA synthesis kit and subjected to DNA sequencing on a Roche 454 Titanium platform. An initial 300,000 sequences were assembled into 3,822 core contiguous sequences using the Genomics Workbench from CLCbio. These B. tabaci ‘Asia 1’ contigs were analysed in detail. In particular, they were compared to a previously published dataset of 1017 contigs from the B. tabaci B biotype (Leshkowitz D et al (2006) Whitefly (Bemisia tabaci) genome project: analysis of sequenced clones from egg, instar, and adult (viruliferous and non-viruliferous) cDNA libraries. BMC Genomics 7:79). Primary sequence information and contig assemblies from whitefly biotypes were integrated within a unified B. tabaci transcriptome database and annotated against other genomes using BLAST, BLAST2GO, GOanna and Exonerate. htNGS gives good sequence coverage of abundantly expressed B. tabaci genes. Hsp90, in particular, was found to be abundantly expressed in B. tabaci biotype Asia 1 whitefly.

FIG. 3a shows the results of a nucleotide alignment of B. tabaci biotype ZHJ1, biotype B (DQ093381.1), biotype Q and biotype Asia 1 hsp90. The alignment identifies polymorphisms (nucleotide variations) between the biotypes at the 111, 129, 402, 408, 420, 429, 444, 480, 621, 645, 646, 669, 708, 729, 741, 747, 771, 832, 843, 948, 963, 999, 1056, 1092, 1101, 1131, 1134, 1164, 1185, 1189, 1269, 1292, 1368, 1381, 1434, 1484, 1488, 1542, 1578, 1614, 1635, 1647, 1707, 1716, 1728, 1733, 1760, 1770, 1776, 1803, 1822, 1863, 1903, 1943, 1947, 1986, 2019, 2043, 2046, 2049, 2061, 2094, 2112 and 2157 positions of SEQ ID NO: 1. In one embodiment, combinations of these polymorphisms are used to identify B. tabaci biotype Asia 1 whitefly DNA. For example, nucleotide C at the 402 position in combination with nucleotide T at the 621 position in SEQ ID NO: 1 identifies B. tabaci biotype Asia 1 DNA.

FIG. 3c shows the results of a nucleotide alignment of B. tabaci biotype ZHJ1, biotype B (EU934241.1), biotype Q and biotype Asia 1 hsp90. The alignment identifies polymorphisms (nucleotide variations) between the biotypes at the 111, 129, 402, 408, 420, 429, 444, 480, 621, 645, 646, 669, 708, 729, 741, 747, 771, 832, 843, 948, 963, 1044, 1048, 1056, 1061, 1090, 1092, 1101, 1131, 1134, 1164, 1185, 1189, 1269, 1292, 1368, 1381, 1434, 1484, 1488, 1542, 1578, 1614, 1635, 1647, 1707, 1716, 1760, 1770, 1776, 1803, 1822, 1863, 1903, 1947, 1953, 1986, 2019, 2043, 2046, 2049, 2055, 2061, 2094, 2112 positions of SEQ ID NO: 1

The alignment of FIG. 3a further provides a number of polymorphisms that, in isolation, identify biotype Asia 1 DNA. In particular, nucleotide T at the 111, 420, 444, 708, 729, 948, 963, 1189, 1647, 1803, 1986 or 2049 position of SEQ ID NO: 1 identifies biotype Asia 1. Alternatively, an equivalent genetic marker of 111T, 420T, 444T, 708T, 729T, 948T, 963T, 1189T, 1647T, 1803T, 1986T or 2049T of SEQ ID NO: 1 identifies biotype Asia 1. Other polymorphisms that, in isolation, identify biotype Asia 1 include: nucleotide C at the 129 or 741 position of SEQ ID NO: 1, nucleotide A at the 1185, 1707, 1822 or 1903 position of SEQ ID NO: 1, or an equivalent genetic marker of 129C, 741C, 1185A, 1707A, 1822A or 1903A.

The genetic polymorphisms in the hsp90 gene serve as a diagnostic test for biotype Asia 1 whitefly. In one embodiment, nucleic acids are extracted (as RNA or DNA) by methods known in the art, from whitefly harvested in the field or from quarantine import testing centres. The nucleotide at one or more of the 111, 129, 402, 408, 420, 429, 444, 480, 621, 645, 646, 669, 708, 729, 741, 747, 771, 832, 843, 948, 963, 999, 1036, 1092, 1101, 1131, 1134, 1164, 1185, 1189, 1269, 1292, 1368, 1381, 1434, 1484, 1488, 1542, 1578, 1614, 1635, 1647, 1707, 1716, 1728, 1733, 1760, 1770, 1776, 1803, 1822, 1863, 1903, 1943, 1947, 1986, 2019, 2043, 2046, 2049, 2061, 2094, 2112 and 2157 positions of SEQ ID NO: 1 is determined directly by sequencing the DNA, indirectly by sequencing cDNA derived from the RNA, or indirectly by sequencing polymerase chain reaction amplification products derived from either the RNA or DNA.

Sequences for the B. tabaci Asia 1 metabolic enzyme enolase at the nucleotide level (SEQ ID NO: 3) and the protein level (SEQ ID NO: 4), together with Asia 1 sequences for the metabolic enzyme triose phosphate isomerase (SEQ ID NO: 5 at the nucleotide level, and SEQ ID NO: 6 at the protein level), the metabolic enzyme fructose bisphosphate aldolase (SEQ ID NO: 7 at the nucleotide level and SEQ ID NO: 8 at the protein level), and a putative vitellogenin (SEQ ID NO: 9) are shown in the accompanying figures.

A diagnostic test for the B. tabaci Asia 1 biotype was developed by deriving PCR products from Asia 1 whitefly RNA using oligonucleotide primers designed to the Asia 1 whitefly vitellogenin mRNA (SEQ ID NO: 9). Sequencing the amplification product showed that it corresponded precisely to the cDNA sequence previously determined for this RNA (SEQ ID NO: 12).

In one embodiment, the amino acid sequence corresponding to the nucleotide sequence SEQ ID NO: 1 was used to identify B. tabaci biotype Asia 1. FIG. 5 shows an amino acid alignment of biotype ZHJ1, B, Q and Asia 1 hsp90. The alignment provides polymorphisms at the 224, 439, 503, 586, 595 and 656 positions of SEQ ID NO: 2. Combinations of these polymorphisms are used to identify biotype Asia 1. For example, amino acid E at the 224 position in combination with F at the 503 position and N at the 586 position in SEQ ID NO: 2 identifies a biotype Asia 1 polypeptide.

In a further embodiment, a B. tabaci Asia 1 biotype hsp90 protein is identified by protein sequencing a whitefly hsp90 protein and comparing the resulting amino acid sequence to SEQ ID NO: 2. Protein sequencing is by any method known in the art.

Real-time qPCR was used to quantify gene expression in commercially important B. tabaci populations. For example, one vitellogenin gene identified by htNGS was used as a target for real time PCR quantification from purified total RNA. The resulting PCT products were subjected to re-sequencing to confirm observed sequence variations (FIG. 24).

Antibodies or other agents that recognise the biotype Asia 1 polypeptide of SEQ ID NO: 2 were used as a protein-based diagnostic to recognise whitefly hsp90 proteins. A kit comprising the agent is used in the field to identify biotype Asia 1 whitefly. Useful agents are found by screening a library of molecules or compounds—such as antibodies or their fragments, peptides, or drug-like molecules or their fragments. Screening comprises combining the library with a biotype Asia 1 hsp90 protein and detecting binding, if any, to identify ligands that bind the protein. Alternatively, a polyclonal antibody specific to the polypeptide of SEQ ID NO: 2 is produced by generating an antigen based on the polypeptide of SEQ ID NO: 2.

Agrochemicals or other agents that recognise the biotype Asia 1 polypeptide of SEQ ID NO: 2, SEQ ID NO: 4, SEQ ID NO: 6 or SEQ ID NO: 8 are also used as a protein-based diagnostic to recognise whitefly hsp90 proteins, inhibit them, or arrest the growth of the whitefly that harbour them. A chemical mixture including the agent is used in the field to arrest biotype Asia 1 whitefly infestations.

### EXAMPLE 1

### Sequencing Whitefly RNA and DNA

RNA was extracted from whitefly using chemicals such as guanidinium thiocyanate, lithium chloride or other substances known to preserve RNA integrity. Complementary DNA (cDNA) was produced, either from the extracted RNA or from mRNA preparations refined from it, using reverse transcriptase or similar enzymes. The resulting cDNA was subjected to standard DNA sequencing reactions, for example using dideoxy-terminator chemistry or oligonucleotide primer-extension techniques. The sequences obtained are compared to known whitefly sequences to determine whether or not the whitefly was biotype Asia 1 (see FIG. 3). In one embodiment, high-throughput next generation sequencing is used to determine nucleotide sequences.

PCR-based techniques using primers designed from the sequences of the present invention were also used to produce DNA by selective amplification of segments of RNA containing informative polymorphisms from total RNA preparations isolated from target insects such as whitefly. Results are illustrated in FIGS. 19, 20, 21, 22 and 23 for biotype Asia 1 vitellogenin RNA.

A similar approach is used to identify the disclosed polymorphisms within whitefly genomic DNA. In this case, DNA is isolated by known techniques, and either sequenced directly, or via PCR products from which sequences can be determined. Determination of biotype Asia 1 is by comparison with the sequences of the present invention.

### EXAMPLE 2

### Antibody Production

Peptide sequences encompassing known Asia 1 polymorphisms are chemically synthesised using known techniques—such as solid phase peptide synthesis. Following an inoculation regime, these peptides are injected into animals, such as rabbits or goats, together with appropriate immunological enhancers, and serial examination for an immune response to the immunizing peptide is carried out. Polyclonal sera are produced, from which antibody fractions and fragments are obtained and purified—by, for example, affinity chromatography on Protein A or immobilised peptides.

### EXAMPLE 3

### Antibody Assay

Whitefly proteins such as hsp90 are purified either from native whitefly or as recombinant proteins from a transgenic organism containing the gene for the whitefly protein, and immobilised to a surface such as a micotitre plate or a bead. Alternatively synthetic peptides derived from target proteins are immobilised. Antibodies specific to the Asia 1 proteins, produced as described in Example 2, are labelled and used to detect the presence of these proteins. In one embodiment, an ELISA-based kit is produced to detect the presence of Asia 1 proteins.

### EXAMPLE 4

### Screening a Library for Agents that Bind Polypeptide of SEQ ID NO: 2

Whitefly proteins, including hsp90, are purified either from native whitefly or as recombinant proteins from a transgenic organism containing the gene for the whitefly protein. An assay for ligands binding to the proteins is produced. A preferred assay is a functional assay measuring enzyme activity. Another suitable assay is a binding assay, such as the biophysical technique of Surface Plasmon Resonance. The assay detects agents of appropriate size which bind to the protein. Using these methods, synthetic chemicals are iteratively produced and tested to identify a chemical with utility as an agrochemical.

### EXAMPLE 5

### Protein Sequencing Whitefly Hsp90

As an extension to Example 1, whitefly proteins, including hsp90, are purified from native whitefly and subjected to protein sequencing. A preferred method is sequencing by mass spectrometry. The resulting protein sequence is compared to known sequences of whitefly biotypes to identify biotype Asia 1 whitefly.

1. A method of identifying the biotype of a whitefly comprising determining a gene, polypeptide, or protein sequence of the whitefly.

2. A method as claimed in claim 1 wherein the whitefly is a Bemisia tabaci biotype Asia 1 whitefly.

3. A method as claimed in claim 1 or claim 2 comprising using at least one of:
(a) an hsp90 gene, polypeptide or protein,
(b) an enolase gene, polypeptide or protein,
(c) a fructose bisphosphate aldolase gene, polypeptide or protein,
(d) a triose phosphate isomerase gene, polypeptide or protein, or
(e) a vitellogenin gene, polypeptide or protein,
of the whitefly.

4. A method as claimed in claim 3 comprising reacting at least one of:
(a) the hsp90 protein with an agent that recognises the polypeptide of SEQ ID NO: 2,
(b) the enolase protein with an agent that recognises the polypeptide of SEQ ID NO: 4,
(c) the fructose bisphosphate aldolase protein with an agent that recognises the polypeptide of SEQ ID NO: 6, or
(d) the triose phosphate isomerase protein with an agent that recognises the polypeptide of SEQ ID NO: 8,

5. A method as claimed in claim 4 wherein the agent is an antibody or an agrochemical.

6. A method as claimed in claim 3 comprising at least one of:
(a) determining the amino acid sequence of the hsp90 protein of the whitefly and comparing the amino acid sequence to SEQ ID NO: 2.
(b) determining the amino acid sequence of the enolase protein of the whitefly and comparing the amino acid sequence to SEQ ID NO: 4.
(c) determining the amino acid sequence of the fructose bisphosphate protein of the whitefly and comparing the amino acid sequence to SEQ ID NO: 6.
(d) determining the amino acid sequence of the triose phosphate protein of the whitefly and comparing the amino acid sequence to SEQ ID NO: 8.

7. A method as claimed in claim 3 comprising investigating a nucleotide sequence or corresponding amino acid sequence of at least one of:
(a) the hsp90 gene of the whitefly,
(b) the enolase gene of the whitefly,
(c) the fructose bisphosphate aldolase gene of the whitefly,
(d) the triose phosphate isomerase gene of the whitefly, or
(e) the vitellogenin gene of the whitefly.

8. A method as claimed in claim 7 wherein the nucleotide sequence of the hsp90 gene that is investigated is one or more of the 111, 129, 402, 408, 420, 429, 444, 480, 621, 645, 646, 669, 708, 729, 741, 747, 771, 832, 843, 948, 963, 999, 1056, 1092, 1101, 1131, 1134, 1164, 1185, 1189, 1269, 1292, 1368, 1381, 1434, 1484, 1488, 1542, 1578, 1614, 1635, 1647, 1707, 1716, 1728, 1733, 1760, 1770, 1776, 1803, 1822, 1863, 1903, 1943, 1947, 1986, 2019, 2043, 2046, 2049, 2061, 2094, 2112 and 2157 positions of SEQ ID NO: 1.

9. A method as claimed in claim 3 wherein a B. tabaci biotype Asia 1 hsp90 gene is identified by the presence of nucleotide T at the 111, 420, 444, 708, 729, 948, 963, 1189, 1647, 1803, 1822, 1986 or 2049 position of SEQ ID NO: 1, or the presence of an equivalent genetic marker of 111T, 420T, 444T, 708T, 729T, 948T, 963T, 1189T, 1647T, 1803T, 1822T, 1986T or 2049T of SEQ ID NO: 1.

10. A method as claimed in claim 3 wherein an hsp90 nucleotide sequence is investigated using an oligonucleotide that hybridises with SEQ ID NO: 1.

11. A method as claimed in claim 7 wherein the amino acid sequence that is investigated is one or more of the 224, 439, 503, 586, 595 and 656 positions of SEQ ID NO: 2.

12. A method as claimed in claim 11 wherein a biotype Asia 1 hsp90 polypeptide is identified by the presence of glutamic acid at the 224 position of SEQ ID NO: 2 combined with phenylalanine at the 503 position of SEQ ID NO: 2 and asparagine the 586 position of SEQ ID NO: 2.

13. A kit for identifying B. tabaci biotype Asia 1 comprising at least one of:
(a) means for detecting nucleotide T at the 111, 420, 444, 708, 729, 948, 963, 1189, 1647, 1803, 1822, 1986 or 2049 position of SEQ ID NO: 1
(b) means for detecting nucleotide A at the 1185, 1707, 1822 or 1903 position of SEQ ID NO: 1
(c) means for detecting nucleotide C at the 129 or 741 position of SEQ ID NO: 1
(d) means for detecting the presence of a protein derived from the polypeptide sequence of SEQ ID NO: 2, SEQ ID NO: 4, SEQ ID NO: 6 or SEQ ID NO: 8.

14. A kit as claimed in claim 13 wherein the means of (a)-(c) is one or more oligonucleotide.

15. A kit as claimed in claim 13 wherein the means of (d) is an antibody.

16. An antibody or fragment thereof which specifically binds to a polypeptide, the polypeptide comprising: at least 10 contiguous amino acids from the amino acid sequence of SEQ ID NO: 2, SEQ ID NO: 4, SEQ ID NO: 6 or SEQ ID NO: 8, or an immunogenic fragment of the amino acid sequence of SEQ ID NO: 2, SEQ ID NO: 4, SEQ ID NO: 6 or SEQ ID NO: 8.

17. (canceled)

18. A method of screening a library of molecules or compounds to identify a ligand which binds the polynucleotide of one or more of SEQ ID NO: 2, SEQ ID NO: 4, SEQ ID NO: 6 or SEQ ID NO: 8, comprising detecting hybridisation complexes.

19. A method as claimed in claim 18 wherein the ligand is an agrochemical.

20. A method as claimed in claim 2 comprising using at least one of:
(a) an hsp90 gene, polypeptide or protein,
(b) an enolase gene, polypeptide or protein,
(c) a fructose bisphosphate aldolase gene, polypeptide or protein,
(d) a triose phosphate isomerase gene, polypeptide or protein, or
(e) a vitellogenin gene, polypeptide or protein,
of the whitefly.


SEQUENCE LISTING (12 sequences)

SEQ ID NO: 1 - DNA, 2163 bp, Bemisia tabaci
  misc_feature: Asia 1 biotype hsp90
atgccagaagacgttaacatggaacaggctgagaccttcgtatttcaagctgagattgctcagcttatgagcttgattgtcaacacattctactcaaacaaagaaatctttttgcgagaattgatctccaactcatcagatgccttggacaaaatccgttacgaatctctaacagacccatcccgtttggaaagtggcaaagaactttacatcaagatcattcccaacaagaatgacagaactctcaccatcattgatactggaattggaatgaccaaggctgaccttgtaaacaacttgggaacaatcgccaaatctggaactaaagccttcatggaggccctcgctgctggtgctgacatctctatgatcggtcagtttggtgtaggtttctactcagctttcctcgtcgctgacactgtcacagttgtctcaaaacacaatgatgacgaacagtatgtctgggagtcctctgccggtggatctttcacaatcaaatctgaccatggagaacctctcggccgtggaaccaagatcatcatgcacatgaaggaagacatgactgaattcctggaggagcgcaagatcaaagatatcgtcaagaagcactcccagttcattggctatcccatcaaactgctcgtcgagaaggagcgagacaaggaacttagtgaagacgaagaggaagaagaagtgaagaaggaagataaagaagaaaaagaagaagataaggatacacccaagattgaagatgtagaagatgaagaagaaggtaaagagaagaaaaagaagaagaagacaatcaaagaaaagtatactgaagatgaagagttgaacaagacaaaacccatctggacccgcaacccagatgacatcacaacagaagagtacggagagttctacaagtccctcacaaacgactgggaagaccacctagctgtcaaacatttctctgttgaaggtcagctggaattcaaagccttattgttcgtccctcgtcgtgctccattcgacctgttcgagaacaaaaagaagaagaacaacatcaaactgtacgtcagacgagttttcatcatggacaactgcgaagacctcatccctgaatacctgaacttcatcaagggtgtcgttgacagcgaggacttgcccctgaacatttctcgtgaaatgctccaacagaacaaaatcttgaaagtcatccgcaagaacttggtcaagaaatgcttggaacttttcgaagagctggcagaagacaaagagaacttcaagaaattctacgaacagttcagcaagaatttgaaattaggaatccacgaagatactcaaaataggaagaaattggcagacctcctgaggtaccagacttcggccactggagacgatgtctgctcatttaaagattatgtagctcgtatgaaagagaaccagaagcacatctactacatcactggtgaaagcaaagatcaagtagctaactcctcatttgtcgagcgagtcaagaaacgcggttttgaagtaatctacatgaccgaacccatcgatgaatatgtagtccagcaaatgaaagactacgacggtaagaacctggtctcagtcacgaaagaaggattagagctgcctgaggacgaagaagaaaagaagaaatatgaggaagacaaagttaagttcgaaaccctctgcaaggtaatgaaagatatccttgacaaaaaagtagagaaagttattgtaagtaacaggttagtcgaatccccatgctgtatcgtcacatcgcagtacggctggacagcaaacatggaacgtattatgaaggctcaagcgctcagagactcttcgacaatgggctacatggcagccaagaaacatctggaaatcaaccctgaccaccctgtcatggatgctctcagagtgaaagcagaagctgagaagaacgacaagtcagtgaaagatcttgttatgctgttgttcgagacggcccttctgtcctctggttttgctcttgaagatcctcaagtccatgccggccgcatccaccgaatgatcaaactcggtttaggtattgacgaagacgagccagtcatggttgaagaggagaagccagacacagccatgcccgcagctgacggagatgcagaagatgcttcaaggatggaagaagttgattaa

SEQ ID NO: 2 - protein, 720 aa, Bemisia tabaci
  MISC_FEATURE: Asia 1 biotype hsp90
MPEDVNMEQAETFVFQAEIAQLMSLIVNTFYSNKEIFLRELISNSSDALDKIRYESLTDPSRLESGKELYIKIIPNKNDRTLTIIDTGIGMTKADLVNNLGTIAKSGTKAFMEALAAGADISMIGQFGVGFYSAFLVADTVTVVSKHNDDEQYVWESSAGGSFTIKSDHGEPLGRGTKIIMHMKEDMTEFLEERKIKDIVKKHSQFIGYPIKLLVEKERDKELSEDEEEEEVKKEDKEEKEEDKDTPKIEDVEDEEEGKEKKKKKKTIKEKYTEDEELNKTKPIWTRNPDDITTEEYGEFYKSLTNDWEDHLAVKHFSVEGQLEFKALLFVPRRAPFDLFENKKKKNNIKLYVRRVFIMDNCEDLIPEYLNFIKGVVDSEDLPLNISREMLQQNKILKVIRKNLVKKCLELFEELAEDKENFKKFYEQFSKNLKLGIHEDTQNRKKLADLLRYQTSATGDDVCSFKDYVARMKENQKHIYYITGESKDQVANSSFVERVKKRGFEVIYMTEPIDEYVVQQMKDYDGKNLVSVTKEGLELPEDEEEKKKYEEDKVKFETLCKVMKDILDKKVEKVIVSNRLVESPCCIVTSQYGWTANMERIMKAQALRDSSTMGYMAAKKHLEINPDHPVMDALRVKAEAEKNDKSVKDLVMLLFETALLSSGFALEDPQVHAGRIHRMIKLGLGIDEDEPVMVEEEKPDTAMPAADGDAEDASRMEEVD

SEQ ID NO: 3 - DNA, 1302 bp, Bemisia tabaci
  misc_feature: Asia 1 biotype enolase
atgccgatctcaaaagttttctctcgccaaatcttcgactcccgtggaaacccaactgttgaggttgatgttgtaactgagttaggtttgttccgtgctgctgttccatctggtgccagcactggtatccatgaggccctggagttgcgcgataacaacaagaacgcctatttgggtaaaggagtctcaaaagctgtcgaaaacgtcaacaaaattattgcaccagaactgttgaaagcgaacctggaagtaacaaaccaaaaagagattgatgagttccttttgaagttggacggcaccgaaaacaaatccaaattgggtgctaatgcaatccttggtgtttctctggctgttgctaaggctggagctgccaagaaaggagttcctctgtacaagcacattgctgacctcgctggcaatgacaacatcattttaccagtccctgctttcaatgtcatcaacggaggttcgcatgccggtaacaaattggccatgcaggagttcatgatccttccaacaggtgcatcctcattctcagaagctatgaaaatcggcgctgaagtgtaccacaatctcaagaaagtgatcaacgccaagtttggtttggatgctaccgctgtaggtgatgagggaggttttgcgcccaacatcctcgataacaaggaagggctgaggttgatcattgaagctattgacaaggctggctactccggtaaagtcgatattggaatggatgttgcagcatctgaattttacaaggaaggtttgtacgatttggacttcaaaaacccaaaatcagacaaaagccagtggatcaacagcgacaagctgaccaacatctacgaagacttcatcaaagaataccccatcgtctccatcgaggatcccttcgaccaggaccattgggaagcttggagcaacatcaccgccaaaaccaatatccagatcgtcggagatgatttgactgtgacgaatcccaagcgaatccaaacagcagttgaaaagaaggcatgtaactgccttttactcaaggtcaaccagatcggttctgtaactgaatccatcgccgctcatctgctggccaagaagaatggttggggaaccatggtctctcataggtcaggcgaaacagaagacactttcatcgccgatttggtcgttggattgagcacaggacagattaaaactggagctccttgcagatctgagcgtctcgccaaatacaaccaaatcctccgaattgaggaagaactaggagcagctgccaaattcgctggcaagaatttccgtcatcctgtctaa

SEQ ID NO: 4 - protein, 433 aa, Bemisia tabaci
  MISC_FEATURE: Asia 1 biotype enolase
MPISKVFSRQIFDSRGNPTVEVDVVTELGLFRAAVPSGASTGIHEALELRDNNKNAYLGKGVSKAVENVNKIIAPELLKANLEVTNQKEIDEFLLKLDGTENKSKLGANAILGVSLAVAKAGAAKKGVPLYKHIADLAGNDNIILPVPAFNVINGGSHAGNKLAMQEFMILPTGASSFSEAMKIGAEVYHNLKKVINAKFGLDATAVGDEGGFAPNILDNKEGLRLIIEAIDKAGYSGKVDIGMDVAASEFYKEGLYDLDFKNPKSDKSQWINSDKLTNIYEDFIKEYPIVSIEDPFDQDHWEAWSNITAKTNIQIVGDDLTVTNPKRIQTAVEKKACNCLLLKVNQIGSVTESIAAHLLAKKNGWGTMVSHRSGETEDTFIADLVVGLSTGQIKTGAPCRSERLAKYNQILRIEEELGAAAKFAGKNFRHPV

SEQ ID NO: 5 - DNA, 1095 bp, Bemisia tabaci
  misc_feature: Asia 1 biotype fructose 1,6-bisphosphate aldolase coding sequence
atgaccacctatttcaactaccctcccgctgaattgcaagaggagcttaagaaaatcgcgcaggccatcgtagcccctggaaagggtatccttgccgccgacgaatccacctctaccatcggcaaacgtttcaaggacatcggcgccgaaaacaacgaggagaacagacgtcagtaccgtcagttgttattcacaacagacaaagctctcgctgataacatctccggtgttatcttgttccacgagtctctttaccagaaggctgatgatggcactccattccccgtcctattgaaacagaaaggaattattcccggaatcaaggttgataagggtgttgtcaaccttttcggatctgaaggagagacaaccacccaaggtttggatgatctggctgaacgttgcgctcaatacaaaaaggacggtgctgacttcgcaaaatggcgttgtgtcttgaaaattggaaagaacacaccctcctaccaatccatccttgaaaatgccaatgtccttgcccgctatgcctccatctgtcaggccaaccgcatcgtccctattgtcgaacctgaggttcttcccgacggtgagcacgatttggaccgcgcccagaaggttacagaaactgttctagctgctgtttacaaggcactcaatgaccaccatgtatatctggaaggcacactcctgaagccaaacatggtgaccgctggtcagtcatgcccaaccaaatacactccagctgatgttgcaaaggccacagtcaccgccttccaacgcacagtccctgttgccgttccaggagttaccttcttatccggaggacagtctgaagaagaagcctctgtccacctagacgctatcaacaaataccccggcaagaaaccatggaccctcaccttcagttacggtcgtgcccttcaggcttccgtgctccgtgcatggggaggcaaagcggagaatgttaaggctgcccaagatgaattactgaagagagccaaggctaatggtgatgcggcactcggaaaatatgttgctggatcagtccaaggacaagctggcgatgcatctctgttcatccaaaaccatgcatactaa

SEQ ID NO: 6 - protein, 364 aa, Bemisia tabaci
  MISC_FEATURE: Asia 1 biotype fructose 1,6-bisphosphate aldolase protein
MTTYFNYPPAELQEELKKIAQAIVAPGKGILAADESTSTIGKRFKDIGAENNEENRRQYRQLLFTTDKALADNISGVILFHESLYQKADDGTPFPVLLKQKGIIPGIKVDKGVVNLFGSEGETTTQGLDDLAERCAQYKKDGADFAKWRCVLKIGKNTPSYQSILENANVLARYASICQANRIVPIVEPEVLPDGEHDLDRAQKVTETVLAAVYKALNDHHVYLEGTLLKPNMVTAGQSCPTKYTPADVAKATVTAFQRTVPVAVPGVTFLSGGQSEEEASVHLDAINKYPGKKPWTLTFSYGRALQASVLRAWGGKAENVKAAQDELLKRAKANGDAALGKYVAGSVQGQAGDASLFIQNHAY

SEQ ID NO: 7 - DNA, 744 bp, Bemisia tabaci
  misc_feature: Asia 1 biotype triose phosphate isomerase coding sequence
atgagtcgaaaattcttcgtcggtggcaactggaaaatgaatggtgacaaagcccaaattgagcagatcgtgcagttcttaaaaacaggacctctggatcctgctgttgaggttgtcgttggagttccagctgtgtatttggagcatgtcaaaaacattttgcccagtaacgtcaaagtctcagcccagaattcctacaaagttcccaaaggagctttcactggtgaaataagtcctgcacaaatcaaggatgttggtgctgactgggttatcttgggtcactctgagaggcgaaacgtttttggagaaaatgatgtgctaatcgctgagaaaacaatccatgcattagaaacaggactgtcagttattgcttgcatcggagaaaaactggaggaaagagaggctggtcagacggaggcagtcgtttatcaacagacaaaagccctagctgataaaattactgactggtcgaatgtagtccttgcttatgaacccgtttgggcgattggaactggaaagacggcaactcctcaacaggctcaagaggttcacaaacaactccgagaatggctcaaagctaatgtatcagaaaaagtggctcaaacattgagaataatctatggagggtcagtaactgctgccaattgtaaagaacttgccaaggaacccgacatagatggatttttggtcggaggagcatctttgaaacctgaatttgttgaaattgtaaatgctcgccaataa

SEQ ID NO: 8 - protein, 247 aa, Bemisia tabaci
  MISC_FEATURE: Asia 1 biotype triose phosphate isomerase protein
MSRKFFVGGNWKMNGDKAQIEQIVQFLKTGPLDPAVEVVVGVPAVYLEHVKNILPSNVKVSAQNSYKVPKGAFTGEISPAQIKDVGADWVILGHSERRNVFGENDVLIAEKTIHALETGLSVIACIGEKLEEREAGQTEAVVYQQTKALADKITDWSNVVLAYEPVWAIGTGKTATPQQAQEVHKQLREWLKANVSEKVAQTLRIIYGGSVTAANCKELAKEPDIDGFLVGGASLKPEFVEIVNARQ

SEQ ID NO: 9 - DNA, 118 bp, Artificial sequence
  Synthetic oligonucleotide
cttgcttctctgtcaatgctatccctacttgcagatatccatacaagccccaaggtggagcttacaaggagattgacttctactgtgtctcaagaaattctgaggaagctcgccacta

SEQ ID NO: 10 - DNA, 20 bp, Artificial sequence
  Synthetic oligonucleotide
cttgcttctctgtcaatgct

SEQ ID NO: 11 - DNA, 21 bp, Artificial sequence
  Synthetic oligonucleotide
ttctgaggaagctcgccacta

SEQ ID NO: 12 - DNA, 53 bp, Artificial sequence
  Synthetic oligonucleotide
aagccccaaggtggagcttacaaggagattgacttctactgtgtctcaagaaa